SEQUENCE LISTING (3 sequences)

SEQ ID NO: 1 - DNA, 762 bp, Artificial Sequence
  Nucleotide sequence of rsib orf.1266
gtgaagaaagatgagtctcccaaaagacaaaaaattgaccttagtcgaaatgatttattgttgcagaaaattaagaataatgatcctgaaattgtttgtgtagaactaccttatgaagaagatagtagttttttgtcaattgtaattaaaactttagagaaaaatactgtagtaaaagaaattgatttatatggcagcaaccttagtaacgatgatataaaaaatctaagtaaaattgtaaagttaaatcaaatatcacatttaaatctgaattctacttctttagatagtgaaaaaataaaaattcttattgatgctttagcagataatacctctcttgaatatttatctttaaattctattgcgctgaaaatagaagatttaagaatattaatagatgatattaaaaaccaccctaatttaagttctttagcattaggtagtagtgaagtaggaaataaaggagcagcaattattagtgagttgtttcattcaaaactaccgttaaaatccttagatataggacaaatgaatattactgccgaagggatagacgttatcgcaaataatattagcagtgcaaaattaatgtcattaaatttagggtataataaccttagaaacgagggagtcataaaattactgcatgctttaatggctaatcaatatattaaagaattaatattaaatgaaacaagaatctccgaaaaaagtgcagaagtaattgaaggttttttaacctcacgcctattgatactttag

SEQ ID NO: 2 - protein, 253 aa, Artificial Sequence
  Amino acid sequence of rsib orf.1266
MKKDESPKRQKIDLSRNDLLLQKIKNNDPEIVCVELPYEEDSSFLSIVIKTLEKNTVVKEIDLYGSNLSNDDIKNLSKIVKLNQISHLNLNSTSLDSEKIKILIDALADNTSLEYLSLNSIALKIEDLRILIDDIKNHPNLSSLALGSSEVGNKGAAIISELFHSKLPLKSLDIGQMNITAEGIDVIANNISSAKLMSLNLGYNNLRNEGVIKLLHALMANQYIKELILNETRISEKSAEVIEGFLTSRLLIL

SEQ ID NO: 3 - protein, 257 aa, Artificial Sequence
  LRR Domain of Human NOD1
FVLHHFPKRLALDLDNNNLNDYGVRELQPCFSRLTVLRLSVNQITDGGVKVLSEELTKYKIVTYLGLYNNQITDVGARYVTKILDECKGLTHLKLGKNKITSEGGKYLALAVKNSKSISEVGMWGNQVGDEGAKAFAEALRNHPSLTTLSLASNGISTEGGKSLARALQQNTSLEILWLTQNELNDEVAESLAEMLKVNQTLKHLWLIQNQITAKGRAQLADALQSNTGITEICLNGNLIKPEEAKVYEDEKRIICF

(19) United States
(12) Patent Application Publication
(10) Pub. No.: US 2005/0026218 A1
(43) Pub. Date: Feb. 3, 2005

(54) Protein interaction mapping

(75) Inventor: Joel Malek, Beverly, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Agencourt Bioscience Corporation, Beverly, MA (US)

(21) Appl. No.: 10/858,837

(22) Filed: Jun. 1, 2004

Related U.S. Application Data
(60) Provisional application No. 60/474,703, filed on May 30, 2003.

(51) Int. Cl. G01N 33/53; C07H 21/04; C12N 1/21; C07K 14/195

(52) U.S. Cl. 435/7.1; 435/69.1; 435/320.1; 435/252.3; 530/350; 536/23.7

(57) ABSTRACT

The present invention relates to isolated nucleic acids that encode polypeptides that interact with T4SS (referred to herein as “T4SS interactor nucleic acids” and “T4SS interactor polypeptides”) and complements, orthologs, portions and variants thereof. The present invention also relates to isolated T4SS interactor polypeptides, orthologs and portions thereof, and antibodies or antigen binding fragments thereof that specifically bind a T4SS interactor polypeptide. The present invention also relates to constructs and host cells comprising the nucleic acid molecules described herein. In addition, the present invention relates to uses of the nucleic acid and polypeptide molecules provided herein.

### RELATED APPLICATION

This application claims the benefit of U.S. Provisional Application No. 60/474,703, filed May 30, 2003. The entire teachings of the above application is incorporated herein by reference.

### BACKGROUND OF THE INVENTION

Numerous pathogens, such as the Spotted Fever Group pathogens (e.g., Rickettsiae), are obligate intracellular human pathogens that utilize the Type IV Secretion System (T4SS) for delivery of effector molecules to cells of the eukaryotic host organism. Many of these pathogens invade endothelial cells and cause lysis after large amounts of progeny have accumulated. Little is known about specific virulence factors and the mode of pathogenicity of such pathogens. Studies have been conducted on interactions among subunits of the T4SS complex in several microbes (Ward, D. V., et al., Proc. Natl. Acad. Sci., USA, 99:11493-11400 (2002); Ohashi, N., et al., Infect. Immun., 70:2128-2138 (2002); Das, A., et al., J. Bacteriol., 182:758-763 (2000); Christie, P. J., et al., Mol. Microbiol., 40:294-305 (2001)). While interactions among the subunits have been characterized, interactions between the T4SS complex and other proteins, such as secreted effectors, have not been well characterized.

Thus, a greater understanding of interactions between the T4SS complex and other proteins would be useful in diagnosing and treating the conditions caused by such pathogens.

### SUMMARY OF THE INVENTION

The present invention relates to isolated nucleic acids that encode polypeptides that interact with T4SS (referred to herein as “T4SS interactor nucleic acids” and “T4SS interactor polypeptides”) and complements, orthologs, homologs, portions and variants thereof. The present invention also relates to isolated T4SS interactor polypeptides, orthologs, homologs, portions and variants thereof, and antibodies or antigen binding fragments thereof that specifically bind a T4SS interactor polypeptide. The present invention also relates to constructs and host cells comprising the nucleic acid molecules described herein. In addition, the present invention relates to uses of the nucleic acid and polypeptide molecules provided herein.

The present invention relates to an isolated nucleic acid molecule comprising SEQ ID NO: 1. In one embodiment, the isolated nucleic acid molecule is the complement of SEQ ID NO: 1. In another embodiment, the isolated nucleic acid molecule encodes an amino acid sequence comprising SEQ ID NO: 2. In yet another embodiment, the isolated nucleic acid molecule comprises a sequence that hybridizes under highly stringent conditions to SEQ ID NO: 1 or a complement of SEQ ID NO: 1. In a particular embodiment, the isolated nucleic acid molecule comprises a sequence that hybridizes under highly stringent conditions to a complement of SEQ ID NO: 1 and encodes a rsib_orf. 1266 polypeptide.

The present invention also relates to a probe comprising a nucleotide sequence that comprises at least about 40 nucleotides of SEQ ID NO: 1. In a particular embodiment, the isolated nucleic acid comprises at least about 40 nucleotides, wherein the sequence is hybridizable to SEQ ID NO: 1.

The present invention is also directed to an isolated polypeptide encoded by a nucleic acid comprising SEQ ID NO: 1. In one embodiment, the isolated polypeptide has an amino acid sequence comprising SEQ ID NO: 2.

Expression constructs comprising SEQ ID NO: 1 are also encompassed by the present invention. In one embodiment, SEQ ID NO: 1 is operably linked to a regulatory sequence.

The present invention is also related to a host cell comprising isolated nucleic acid described herein.

The present invention is also directed to a method of producing a Rickettsia sibirica rsib_orf.1266 polypeptide comprising culturing the host cell under conditions in which the Rickettsia sibirica rsib_orf.1266 polypeptide is produced. The method can further comprise isolating the Rickettsia sibirica rsib_orf.1266 polypeptide from the cell. Accordingly, the invention is also directed to an isolated Rickettsia sibirica rsib_orf1266 polypeptide produced by the method.

The present invention is also directed to an antibody (e.g., polyclonal, monoclonal) or antigen binding fragment thereof that specifically binds to a Rickettsia sibirica rsib_orf.1266 polypeptide, wherein the Rickettsia sibirica rsib_orf.1266 polypeptide is encoded by an isolated nucleic acid that encodes SEQ ID NO: 2.

The present invention is also directed to a method of identifying a nucleic acid that encodes a Rickettsia polypeptide in a sample comprising contacting the sample with a complement of a nucleotide sequence comprising SEQ ID NO: 1 under conditions in which hybridization occurs between the complement and nucleic acid in the sample using high stringency conditions. Nucleic acid which hybridizes to the complement of the nucleotide sequence comprising SEQ ID NO: 1 under high stringency conditions is identified, thereby identifying a nucleic acid that encodes a Rickettsia polypeptide in a sample. In a particular embodiment, the invention relates to a method of identifying a nucleic acid that encodes a Rickettsia sibirica rsib_orf.1266 polypeptide in a sample.

The invention is also directed to a method of identifying a Rickettsia polypeptide in a sample comprising contacting the sample with an antibody or antigen binding fragment thereof that specifically binds to a Rickettsia sibirica rsib_orf.1266 polypeptide wherein the Rickettsia sibirica rsib_orf.1266 polypeptide is encoded by an isolated nucleic acid that encodes SEQ ID NO: 2. The polypeptide which specifically binds to the antibody is then identified, thereby identifying a Rickettsia polypeptide in a sample. In a particular embodiment, the invention relates to a method of identifying a Rickettsia sibirica rsib_orf.1266 polypeptide in a sample.

The present invention also relates to a method of identifying an agent that alters interaction of a polypeptide of a pathogen with a Type IV secretion system (T4SS) polypeptide (e.g., a Rickettsia sibirica polypeptide such as the rsib_orf.1266 polypeptide; a VirD4 polypeptide, a VirB11 polypeptide; aVirB8 polypeptide), wherein the pathogen utilizes the T4SS and the polypeptide has a leucine rich repeat domain, comprising contacting a Rickettsia sibirica rsib_orf.1266 polypeptide having an amino acid sequence comprising SEQ ID NO: 2 and the Type IV secretion system polypeptide under conditions in which the Rickettsia sibirica rsib_orf.1266 polypeptide interacts with the Type IV secretion system polypeptide, with an agent to be assessed. The extent to which Rickettsia sibirica rsib_orf.1266 polypeptide interacts with the Type IV secretion system polypeptide in the presence of the agent to be assessed is then determined, wherein if the extent to which Rickettsia sibirica rsib_orf.1266 polypeptide interacts with the Type IV secretion system polypeptide is altered in the presence of the agent compared to the extent to which Rickettsia sibirica rsib_orf.1266 polypeptide interacts with the Type IV secretion system polypeptide in the absence of the agent, then the agent alters interaction of a polypeptide of the pathogen with the Type IV secretion system polypeptide. In a particular embodiment, the invention is directed to a method of identifying an agent that alters interaction of a polypeptide of a pathogen with a Type IV secretion system (T4SS) polypeptide, wherein the pathogen utilizes the T4SS and the polypeptide has a leucine rich repeat domain comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is then assessed, wherein if apoptosis of the cell is altered compared to the apoptosis of a control cell, then the agent alters interaction of a Rickettsia polypeptide with Type IV secretion system polypeptide.

The invention also relates to a method of identifying an agent that inhibits an interaction of a polypeptide of a pathogen with a Type IV secretion system (T4SS) polypeptide, wherein the pathogen utilizes the T4SS and the polypeptide has a leucine rich repeat domain comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is then assessed, wherein an increase in apoptosis of the cell compared to apoptosis of a control cell indicates that the agent inhibits interaction of a polypeptide of a pathogen with a Type IV secretion system (T4SS) polypeptide.

The present invention is also directed to a method of identifying an agent that enhances an interaction of a polypeptide of a pathogen with a Type IV secretion system (T4SS) polypeptide, wherein the pathogen utilizes the T4SS and the polypeptide has a leucine rich repeat domain comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is then assessed, wherein a decrease in apoptosis of the cell compared to apoptosis of a control cell indicates that the agent enhances interaction of a polypeptide of a pathogen with a Type IV secretion system (T4SS) polypeptide.

The present invention is also directed to a method of treating an infection by a pathogen in an individual, wherein the pathogen utilizes a Type IV secretion system (T4SS), comprising administering to the individual an agent that inhibits interaction of a Rickettsia sibirica rsib_orf.1266 polypeptide with a Type IV secretion system polypeptide. In a particular embodiment, the present invention is directed to a method of treating a Rickettsia infection (e.g., Rickettsia sibirica; Rickettsia prowazekii; Rickettsia conorii; Rickettsia rickettsii; Rickettsia typhi) in an individual comprising administering to the individual an agent that inhibits interaction of a Rickettsia sibirica rsib_orf.1266 polypeptide with a Type IV secretion system polypeptide.

The present invention also relates to a method of inducing an immune response a pathogen in an individual, wherein the pathogen utilizes a Type IV secretion system (T4SS), comprising administering to the individual all or a portion of a Rickettsia sibirica rsib_orf.1266 polypeptide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are schematics of the Bacterial Two-Hybrid system. FIG. 1A is a schematic of the lambda cI fused to the bait protein which dimerizes and binds the lambda operator. FIG. 1B is a schematic showing that a protein interaction between the Bait and Prey protein recruits the RNAP complex, via the RNAP alpha subunit, to the weak promoter site directing transcription of the reporter genes.

FIG. 2 is a schematic of the Functional Shotgun Sequencing Pipeline. (i) Genomic DNA is sheared and cloned into bait and prey vectors. (ii) Randomly selected bait clones are sequenced, the data assembled and the genome annotated (iii) Clones determined to contain fragments of genes expressed in-frame are re-arrayed for screening. A copy of the set is pooled, and the inserts transferred to the prey vector creating the fragment ORF prey library. (iv) Baits from proteins of interest are either screened against the previously created sheared genomic prey library, or the shuttled fragment ORF prey library. Sequencing of positive clones directly from selected colonies is conducted with pBAIT or pPREY specific primers.

FIG. 3 is an alignment of rsib orf.1266 amino acid sequence (SEQ ID NO: 2) to the LRR domain of human NOD1 protein. Human NOD1 from amino acid 697 to the end (SEQ ID NO: 3) was aligned with the full-length rsib_orf1266 using CLUSTALW. Identical amino acids are shaded in black while similar amino acids are shaded in grey.

FIG. 4 is a map of 148S protein interactions. Nodes represent proteins while edges represent an interaction (Ideker, T., et al., Bioinformatics, 18:S233-S240 (2002)). Subunits of the T4SS used as baits are highlighted in red. The inner, full circle represents interactions shared among the subunits. The broken outer circle represents interactions distinct to a given subunit. Transported effectors may more likely be found in the inner circle as they would interact with more than one subunit.

FIG. 5 is the nucleotide sequence (SEQ ID NO: 1) of rsib orf.1266.

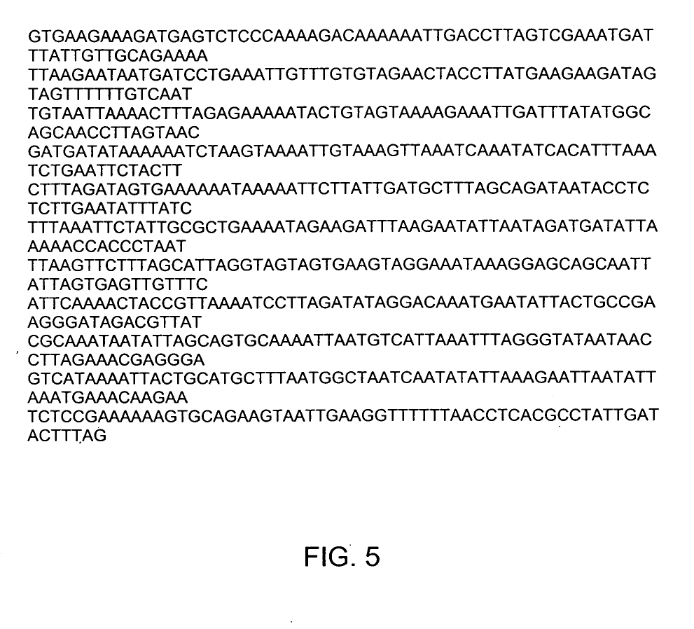

### DETAILED DESCRIPTION OF THE INVENTION

As described herein, a bacterial two-hybrid system was coupled with a whole genome shotgun sequencing approach for microbial genome analysis, addressing a need for large-scale protein interaction analysis. The first large-scale proteomics study using this system, integrating de novo genome sequencing with functional interaction mapping and annotation in a high-throughput format, is described herein. The approach has been applied by shotgun sequencing the genome of Rickettsia sibirica strain 246, an obligate intracellular human pathogen among the Spotted Fever Group Rickettsiae. The bacteria invade endothelial cells and cause lysis after large amounts of progeny have accumulated. Little is known about specific rickettsial virulence factors and their mode of pathogenicity. Analysis of the combined genomic sequence and protein-protein interaction data for a set of virulence related Type IV Secretion System (T4SS) proteins revealed over 250 interactions and provides insight into the mechanism of Rickettsial pathogenicity including evidence of a novel transported host effector.

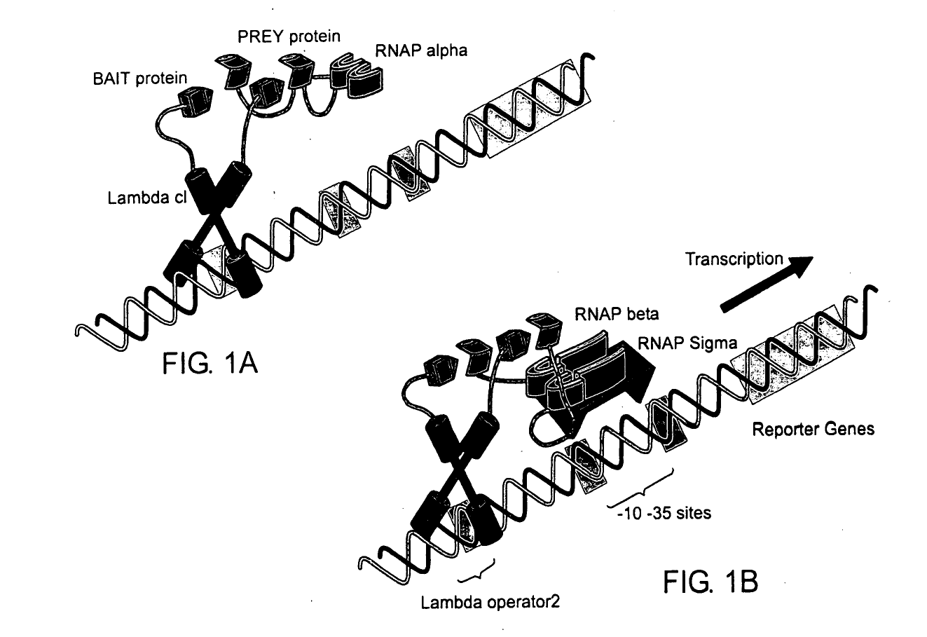

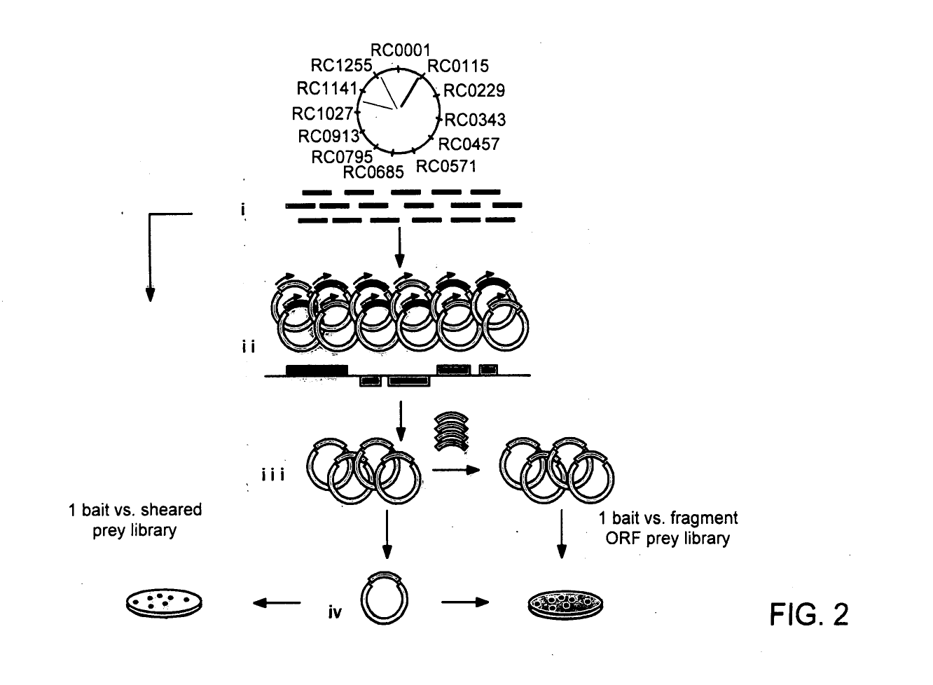

The bacterial two hybrid (B2H) system used in this study described herein was developed by Hochschild and colleagues (Dove, S. L., Nature, 386:627-630 (1997); Dove, S. L.,et al., Genes & Devel., 12:745-754 (1998); Dove, S. L., et al., J. Bacteriol., 183:6413-6421 (2001); Shaywitz, A. J., et al., Mol. Cell Biol., 20:9409-9422 (2000)) and is similar in concept to the standard Y2H system. This method allows for random cloning of fragments because proteins are fused C-terminal to binding or activation domains. Briefly, a protein of interest (the bait) is fused to lambda cI, a DNA binding domain, which binds to a lambda operator sequence, OR2, placed upstream of a weak promoter. In addition, a second protein of interest (the prey) is fused to the RNA Polymerase alpha subunit, an activation domain, which is part of the RNAP holoenzyme (FIG. 1A). If the two proteins of interest interact, RNAP is recruited to the weak promoter causing increased transcription of the downstream reporter genes, Beta-lactamase and Beta-galactosidase (FIG. 1B). Utilizing this system, a process termed “Functional Shotgun Sequencing” in which a shotgun library is constructed in the bait vector, followed by determination of open reading frame (ORF) fragments that are cloned in frame and can be used as baits, was developed (FIG. 2). Since fusion proteins are generated from standard backbone vectors and expressed in E. coli, sequencing of inserts to determine interacting proteins is greatly simplified.

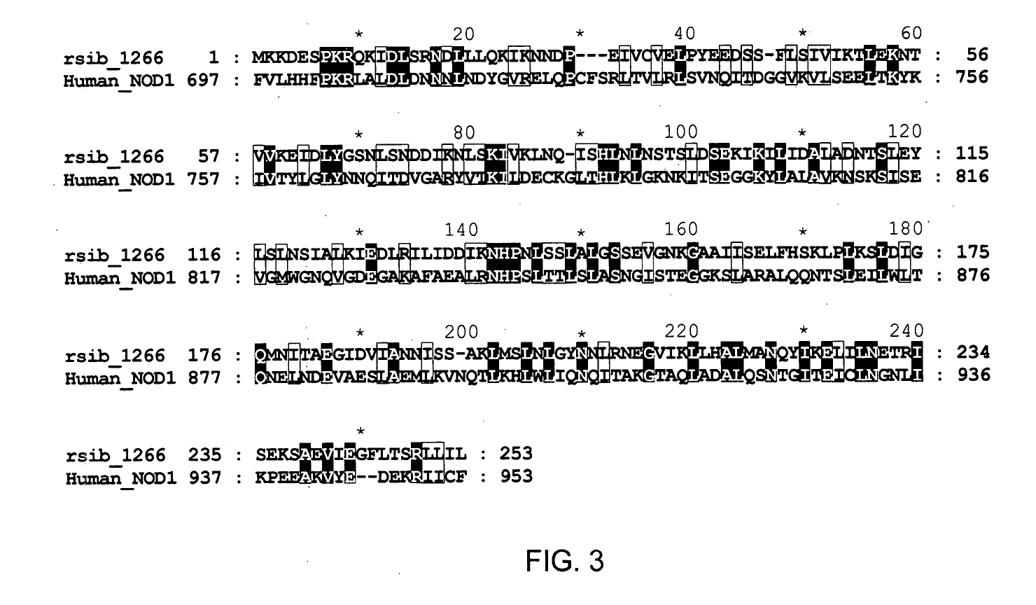

The genome of R. sibirica 246 was subjected to functional shotgun sequencing, assembly, gene identification and automated annotation. Little is known of specific Rickettsial effectors that are secreted during infection (Clifton, D. R., et al., Proc. Natl. Acad. Sci., USA, 95:4646-4651 (1998)). One uncharacterized protein among the T4SS interactors in the screen, rsib_orf.1266, was found to contain a Leucine Rich Repeat (LRR) domain spanning the entire length of the protein, and most similar to the LRR in human NOD proteins (Inohara, N., et al., J. Biol. Chem., 274:214560-14567 (1999)). The protein from rsib_orf.1266 interacts with VirD4, VirB11, and VirB8, all proposed members of the T4SS transfer channel (Christie, P. J., et al., Mol. Microbiol., 40:294-305 (2001)). Interaction with VirD4 is of significance because it is a coupling protein necessary for effector transport in A. tumefaciens and H. pylori (Christie, P. J., et al., Mol. Microbiol., 40:294-305 (2001)). LRR domains have been observed in effectors transported by the type mi secretion system from a variety of intracellular plant and animal pathogens such as R. Solanaacearun (Salanoubat, M., et al., Nature, 415:497-502 (2002)) and Y pestis (Cornelis, G. R., et al., J. Cell Biol., 158:401-408). In addition, LRR domains have been found in the extracellular Intemalin protein of microbes such L. monocytogenes and are involved in host cell internalization of the bacteria (Lecuit, M., et al., Infect. Immun., 65:5309-5319 (1997)). Of particular interest was the high similarity between rsib_orf.1266 and the human NOD family (FIG. 3 ). NOD proteins are involved in bacterial component recognition in human cells through their LRR domain, and have been shown to activate NF-κB and Caspase activity, and subsequent apoptosis, by interacting with RICK (Inohara, N., et al., J. Biol. Chem., 276:2551-2554 (2001)). Crohn's disease, which is associated with mutations in the LRR of a NOD protein, results in cells incapable of bacterial component induced NF-κB activation for apoptosis (Inohara, N., et al., Nat. Rev. Immunol., 3:371-382 (2003)). It has been shown that a fragment spanning the LRR domain of NOD1 alone was able to suppress RICK induced but not TNF alpha induced NF-κB activation (Inohara, N., et al., J. Biol. Chem., 274:14560-14567 (1999)). R. rickettsii, also a member of the Spotted Fever Group, has been shown to modulate NF-κB mediated host cell apoptosis during infection (Clifton, D. R., et al., Proc. Natl. Acad. Sci., USA, 95:4646-4651 (1998)). Despite evidence of their existence, however, no specific host apoptosis modulating effector molecules have been identified in the Rickettsiae. It is proposed herein that rsib_orf.1266, containing an LRR domain, is likely an effector transported by the T4SS, and also that rsib_orf.1266 likely acts as either an internalin, or as a “sink” for bacterial cell wall components, such as LPS and/or peptidoglycan, released during host cell infection. Binding of bacterial components by rsib_orf.1266 would act as a dominant negative NOD mutant disallowing activation of the NOD proteins and subsequent caspase induced apoptosis. This model host molecule mimicry allows for the observations that Rickettsiae activate NF-κB because TNF-alpha induced NF-KB activation could still proceed (Inohara, N., et al., J. Biol. Chem., 274:14560-14567 (1999)). A similar protein to Rsib_orf.1266 was found in R. conorii, but not in R. prowazekii, a Typhus Group Rickettsia, suggesting rsib_orf1266 is a Spotted Fever Group specific effector.

Accordingly, the present invention relates to isolated nucleic acids that encode polypeptides that interact with T4SS (referred to herein as “T4SS interactor nucleic acids” and “T4SS interactor polypeptides”) and complements, orthologs, homologs, portions and variants thereof. The present invention also relates to isolated T4SS interactor polypeptides, orthologs, homologs, portions and variants thereof, and antibodies or antigen binding fragments thereof that specifically bind a T4SS interactor polypeptide. The present invention also relates to constructs and host cells comprising the nucleic acid molecules described herein. In addition, the present invention relates to uses of the nucleic acid and polypeptide molecules provided herein.

In one embodiment, the present invention relates to an isolated nucleic acid sequence comprising SEQ ID NO: 1. In another embodiment, the isolated nucleic acid molecule encodes an amino acid sequence comprising SEQ ID NO: 2.

As used herein “nucleic acid molecule” includes DNA (e.g., cDNA, genomic DNA, a gene), RNA (e.g., mRNA) and analogs thereof. The nucleic acid molecule can be single stranded or double stranded and can be the coding strand (sense strand) or the noncoding strand (antisense strand). The nucleic acid can include all or a portion of the coding strand and can further comprise additional non-coding sequences such as introns and non-coding 5′ and 3′ sequences (e.g., regulatory sequences).

An “isolated” nucleic acid molecule indicates that the nucleic acid molecule is in a form that is distinct from the form in which it occurs in nature. Isolated nucleic acid molecules of the present invention are separated from other nucleic acid molecules which are present in its natural state (e.g., free of sequences which normally flank the nucleic acid in the genome of the organism from which it is derived). In one embodiment, the isolated nucleic acid molecule is part of a composition (e.g., a crude extract). In another embodiment, the isolated nucleic acid molecule is substantially free from the cellular material in which it occurs, and in yet another embodiment, the isolated nucleic acid molecule is purified to homogeneity. Various methods, such as gel electrophoresis or chromatography can be used to identify nucleic acid molecules that are substantially free from cellular materials or purified to homogeneity.

A nucleic acid molecule of the present invention can be isolated using standard recombinant or chemical methods and the sequences provided herein. For example, using all or a portion of SEQ ID NO: 1 as a hybridization probe, a T4SS interactor sequence (e.g., an ortholog of the rsib_orf.1266 nucleic acid sequence) can be isolated using standard hybridization and cloning methods (Sambrook et al., eds., Molecular Cloning: A Laboratory Manual, 2^{nd} ed., Cold Spring Harbor Laboratory, Cold Spring Harbor Laboratory Press, Cold Spring Harbor, N.Y., 1989). A nucleic acid of the invention can be amplified using cDNA, mRNA or genomic DNA as a template and appropriate primers according to standard polymerase chain reaction (PCR) methodology. The amplified nucleic acid can then be cloned into an appropriate vector and characterized using DNA sequence analysis. T4SS interactor nucleic acids can also be prepared using, for example, an automated DNA synthesizer.

In another embodiment, the invention relates to an isolated nucleic acid molecule which is the complement of SEQ ID NO: 1 or a portion thereof. A complement of SEQ ID NO: 1 is a sequence which is sufficiently complementary so that it hybridizes to SEQ ID NO: 1, thereby forming a stable duplex. In a particular embodiment, the complement hybridizes to SEQ ID NO: 1 and encodes a T4SS interactor polypeptide.

The nucleic acid molecule of the invention can comprise a portion of a nucleic acid sequence encoding a T4SS interactor polypeptide. In one embodiment, the portion is a fragment that can be used as a probe or primer. In a particular embodiment, the invention relates to a probe comprising a nucleotide sequence that comprises a portion of SEQ ID NO: 1. In another embodiment, the portion encodes a biologically active portion of a T4SS interactor polypeptide. The portion of a nucleic acid sequence encoding T4SS interactor polypeptide can include all or a portion of the T4SS interactor coding sequence and can further include non-coding sequences such as introns and 5′ and 3′ sequences (e.g., regulatory sequences). The nucleotide sequence of the T4SS interactor provided herein allows for the generation of probes and primers designed for use in identifying and/or cloning T4SS interactor homologues or orthologs from other pathogens (e.g., other Spotted Fever Group pathogen, R. conorii). The portion (e.g., probe/primer) can comprise a substantially purified T4SS interactor oligonucleotide. The portion is generally of a length and composition that hybridizes to all or a characteristic portion of a nucleic acid sequence under stringent conditions. The portion typically comprises a region of nucleotide sequence that hybridizes under stringent conditions to at least about 10, and more particularly about 25, 50, 100, 150, 200, 250, 300, 350, 400, 450, 500, 550, 600, 650, 700 or 750 contiguous nucleotides of the sense or anti-sense sequence of SEQ ID NO:1 or of a naturally occurring mutant of SEQ ID NO:1. In particular embodiments, the portion comprises at least about 40 nucleotides to about 200 nucleotides (e.g., 40 nucleotides, 50 nucleotides, 60 nucleotides, 70 nucleotides, 80 nucleotides 90 nucleotides, 100 nucleotides, 150 nucleotides, 200 nucleotides); about 250 nucleotides to about 450 nucleotides (e.g., about 250 nucleotides, 350 nucleotides, 450 nucleotides); and about 500 nucleotides to about 760 nucleotides (e.g., 550 nucleotides, 650 nucleotides, 750 nucleotides).

Probes based on the T4SS interactor nucleotide sequence described herein can be used to detect transcripts or genomic sequences encoding the same or identical proteins, or splice variants or polymorphisms of the T4SS interactor. A label group (e.g., a radioisotope, a fluorescent compound, an enzyme) can be attached to the probe. Such probes can be used as a part of a diagnostic test kit to assess expression (e.g., aberrant expression) of a T4SS interactor protein in a cell or tissue sample by measuring a level of a T4SS interactor-encoding nucleic acid in a sample from an individual (e.g., detecting T4SS interactor mRNA levels).

A nucleic acid fragment encoding a “biologically active portion of T4SS interactor” can be prepared by isolating a portion of SEQ ID NO: 1 which encodes a polypeptide having a T4SS interactor biological activity, expressing the encoded portion of T4SS interactor protein (e.g., by recombinant expression in vitro) and assessing the activity of the encoded portion of T4SS interactor. A biologically active portion of T4SS interactor includes a portion which retains at least one biological activity of the T4SS interactor polypeptide described herein. Biological activities of the T4SS interactor polypeptide described herein include, for example, interaction with one or more members of the T4SS transfer channel (e.g., VirD4, VirB11 and/or VirB8); internalin activity (acting as a “sink” for bacterial components); activity as a dominant negative mutant disallowing or inhibiting activation of the NOD proteins and subsequent capase induced apoptosis.

The invention further encompasses nucleic acid molecules that differ from the nucleotide sequence of SEQ ID NO:1 due to degeneracy of the genetic code and thus encode the same T4SS interactor protein as that encoded by the nucleotide sequence shown in SEQ ID NO:1. For example, the present invention relates to nucleic acid sequence polymorphisms that lead to changes in the amino acid sequences of T4SS interactor which exist within a population (e.g., a population of Spotted Fever Group pathogens). Such genetic polymorphism in the T4SS interactor gene may exist within a population of pathogens due to natural allelic variation. As used herein, the terms “gene” and “recombinant gene” refer to nucleic acid molecules comprising an open reading frame encoding a T4SS interactor polypeptide. Such nucleotide variations and resulting amino acid polymorphisms in T4SS interactor sequences that are the result of natural allelic variation and that do not alter the functional activity of T4SS interactor are within the scope of the invention.

Moreover, nucleic acid molecules encoding T4SS interactor proteins from other species (T4SS interactor orthologs or homologues), which have a nucleotide sequence which differs from that of a R. sibirica T4SS interactor, are within the scope of the invention. Nucleic acid molecules corresponding to natural allelic variants and homologues of the T4SS interactor nucleic acid of the invention can be isolated based on their identity to the R. sibirica nucleic acid sequence disclosed herein using this sequence, or a portion thereof, as a hybridization probe according to standard hybridization techniques under stringent hybridization conditions. In one embodiment, the nucleic acid molecule of the present invention comprises a nucleotide sequence that is at least about 50%, 60%, 70%, 75%, 80%, 85%, 90%, 95% or 99% identical to SEQ ID NO: 1 or a complement thereof.

Accordingly, in another embodiment, an isolated nucleic acid molecule of the invention is at least about 100, 150, 200, 250, 300, 350, 400, 450, 500, 550, 600, 650, 700, 800, 900, 1000 or 1300 nucleotides in length and hybridizes under stringent conditions to the nucleic acid molecule comprising the nucleotide sequence (and in a particular embodiment, the coding sequence) of SEQ ID NO:1 or the complement thereof. In yet another embodiment, the invention relates to an isolated nucleic acid comprising a nucleotide sequence comprising at least about 40 nucleotides to about 200 nucleotides (e.g., 40 nucleotides, 50 nucleotides, 60 nucleotides, 70 nucleotides, 80 nucleotides 90 nucleotides, 100 nucleotides, 150 nucleotides, 200 nucleotides); about 250 nucleotides to about 450 nucleotides (e.g., about 250 nucleotides, 350 nucleotides, 450 nucleotides); and about 500 nucleotides to about 760 nucleotides (e.g., 550 nucleotides, 650 nucleotides, 750 nucleotides), wherein the sequence is hybridizable to SEQ ID NO: 1.

In one embodiment, the nucleic acid molecule hybridizes to the coding sequence of SEQ ID NO: 1. In a particular embodiment, the nucleic acid molecule hybridizes to SEQ ID NO: 1 and encodes a polypeptide that interacts with a subunit of T4SS.

As used herein, the term “hybridizes under stringent conditions” is intended to describe conditions for hybridization and washing under which nucleotide sequences at least 60%, 65%, 70%, 75%, 80%, 85%, 90%, 95% or 99% identical to each other typically remain hybridized to each other. Such stringent conditions are known to those skilled in the art and can be found in Current Protocols in Molecular Biology, John Wiley & Sons, N.Y. (1989), 6.3.1-6.3.6. One example of stringent hybridization conditions is hybridization in 6×sodium chloride/sodium citrate (SSC) at about 45° C., followed by one or more washes in 0.2×SSC, 0.1% SDS at 50-65° C. In one embodiment, an isolated nucleic acid molecule of the invention that hybridizes under stringent conditions to the sequence of SEQ ID NO:1 corresponds to a naturally-occurring nucleic acid molecule. As used herein, a “naturally-occurring” nucleic acid molecule refers to a nucleic acid molecule having a nucleotide sequence that occurs in nature (e.g., encodes a natural protein).

In addition to naturally-occurring allelic variants of the T4SS interactor sequence that may exist in a population of pathogens, it is known in the art that changes can be introduced by mutation into the nucleotide sequence of SEQ ID NO: 1, thereby leading to changes in the amino acid sequence of the encoded T4SS interactor polypeptide, without altering the functional (biological) ability of the T4SS interactor polypeptide. For example, nucleotide substitutions leading to amino acid substitutions at “non-essential” amino acid residues can be made. Alteration of a “non-essential” amino acid residue in the wild-type sequence of T4SS interactor (e.g., the sequence of SEQ ID NO:2) will not affect the biological activity of T4SS interactor polypeptide. Conversely, an “essential” amino acid residue is required for biological activity of T4SS interactor. Therefore, alteration of an essential amino acid in the wild-type sequence of T4SS interactor will affect the biological activity of T4SS interactor. Amino acid residues that are conserved among the T4SS interactor proteins of various species will likely be essential amino acids. Other amino acid residues, however, (e.g., those that are not conserved or only semi-conserved among T4SS interactor of various species) are likely not essential for activity and thus can be altered without altering the biological activity of T4SS interactor.

Accordingly, another aspect of the invention pertains to nucleic acid molecules encoding T4SS interactor polypeptides that contain changes in amino acid residues that are not essential for activity. Such T4SS interactor polypeptides differ in amino acid sequence from SEQ ID NO:2 and retain T4SS interactor biological activity (e.g., interaction with one or more members of the T4SS transfer channel, such as VirD4, VirB11 and/or VirB8). In one embodiment, the isolated nucleic acid molecule includes a nucleotide sequence encoding a protein that includes an amino acid sequence that is at least about 45%, 50%, 60%, 75%, 85%, 95%, or 98% identical to the amino acid sequence of SEQ ID NO:2.

An isolated nucleic acid molecule encoding a T4SS interactor polypeptide having a sequence which differs from that of SEQ ID NO:2 can be created by introducing one or more nucleotide substitutions, additions or deletions into the nucleotide sequence of T4SS interactor nucleic acid molecule (SEQ ID NO:1) such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. A predicted nonessential amino acid residue in T4SS interactor is preferably replaced with another amino acid residue from the same side chain family. Alternatively, mutations can be introduced randomly along all or part of a T4SS interactor coding sequence, and the resultant mutants can be screened for T4SS interactor biological activity to identify mutants that retain activity. Following mutagenesis, the encoded protein can be expressed recombinantly and the activity of the protein can be determined using methods described herein.

A mutant T4SS interactor polypeptide can be assayed for the ability to interact with one or more members of the T4SS transfer channel (e.g.,VirD4, VirB11 and/or VirB8); for internalin activity (acting as a “sink” for bacterial components); for activity as a dominant negative mutant disallowing or inhibiting activation of the NOD proteins and subsequent capase induced apoptosis or a combination of such activities.

The present invention also encompasses antisense nucleic acid molecules, i.e., molecules which are complementary to a sense nucleic acid encoding a T4SS interactor polypeptide, e.g., complementary to the coding strand of a double-stranded cDNA T4SS interactor molecule or complementary to an mRNA T4SS interactor sequence. The present invention also encompasses nucleic acid molecules that are interfering RNA molecules, such as small interfering RNA (siRNA) and short hairpin RNA (shRNA), of a T4SS interactor mRNA (e.g., siRNA or shRNA of rsib_orf.1266). The antisense nucleic acid or interfering nucleic acid can be complementary to an entire T4SS interactor coding strand, or to only a portion thereof, e.g., all or part of the protein coding region (or open reading frame). An antisense nucleic acid or interfering molecule can be antisense or interfering to a noncoding region of the coding strand of a nucleotide sequence encoding T4SS interactor. The noncoding regions (5′ and 3′ untranslated regions) are the 5′ and 3′ sequences which flank the coding region and are not translated into amino acids. The antisense or interfering nucleic acid molecule can be complementary to the entire coding region of T4SS interactor mRNA, but more preferably is an oligonucleotide which is antisense or interfering to only a portion of the coding or noncoding region of T4SS interactor mRNA. An antisense oligonucleotide can be, for example, about 5, 10, 15, 19, 20, 21, 22, 23, 24, 25, 30, 35, 40, 45 or 50 nucleotides in length. An antisense or interfering nucleic acid of the invention can be constructed using procedures known in the art (e.g., using chemical synthesis and enzymatic ligation reactions).

The invention also relates to isolated T4SS interactor protein or polypeptides, and portions (e.g., biologically active portions) thereof. An “isolated” or “purified” (e.g., partially or substantially) polypeptide or biologically active portion thereof is in a form that is distinct from the form in which it occurs in nature. In one embodiment, the polypeptide is part of a composition (crude extract). In another embodiment, the polypeptide is substantially free of cellular material or other contaminating proteins from the cell or tissue source from which the T4SS interactor protein is derived, or substantially free from chemical precursors or other chemicals when chemically synthesized. The language “substantially free of cellular material” includes preparations of T4SS interactor polypeptide (protein) in which the polypeptide is separated from cellular components of the cells from which it is isolated, recombinantly produced or chemically synthesized. Such preparations of T4SS interactor protein have less than about 30%, 20%, 10%, 5% (by dry weight) of chemical precursors or non-T4SS interactor chemicals. Various methods, such as gel electrophoresis or chromatography can be used to identify polypeptides that are substantially free of cellular material. In one embodiment, the present invention relates to an isolated polypeptide encoded by a nucleic acid comprising SEQ ID NO:1. In another embodiment, the present invention relates to an isolated polypeptide having an amino acid sequence comprising SEQ ID NO:2.

The present invention also relates to portions of a T4SS interactor polypeptide. In one embodiment, the portions are biologically active portions of a T4SS interactor polypeptide and include polypeptides comprising amino acid sequences sufficiently identical to or derived from the amino acid sequence of the T4SS interactor polypeptide (e.g., the amino acid sequence shown in SEQ ID NO:2). Biologically active portions include a portion of the full length T4SS interactor polypeptides, and exhibit at least one activity of a T4SS interactor polypeptide (e.g., interaction with one or more members of the T4SS transfer channel (e.g., VirD4, VirB11 and/or VirB8); internalin activity (acting as a “sink” for bacterial components); activity as a dominant negative mutant disallowing or inhibiting activation of the NOD proteins and subsequent capase induced apoptosis.). Typically, biologically active portions comprise one or more domains or regions with at least one activity of the T4SS interactor protein. A biologically active portion of a T4SS interactor protein can be a polypeptide which is, for example, at least about 10, 25, 50, 40, 60, 80, 100, 120, 140, 150, 160, 200 or more amino acids in length. In one embodiment, the portions are biologically active portions of a nucleic acid or protein and include peptides comprising amino acid sequences sufficiently identical to or derived from the amino acid sequence of SEQ ID NO: 1 or SEQ ID NO:2 (e.g., about 10 to about 250 amino acids (e.g., contiguous), about 50 to about 150 amino acids, and about 200 to about 250 amino acids of SEQ ID NO:2). Biologically active polypeptides include one or more identified T4SS interactor domains, e.g., LRR domain. Other biologically active portions can be prepared by recombinant techniques and evaluated for one or more of the functional activities of a native T4SS interactor polypeptide.

Other T4SS interactor polypeptides of the present invention are substantially identical to SEQ ID NO:2, retain the functional activity of the protein of SEQ ID NO:2, yet differ in amino acid sequence due to natural allelic variation or mutagenesis. T4SS interactor polypeptide interact with one or more members of the T4SS transfer channel (e.g.,VirD4, VirB11 and/or VirB8); exhibit internalin activity (acting as a “sink” for bacterial components); and/or exhibit activity as a dominant negative mutant disallowing or inhibiting activation of the NOD proteins and subsequent capase induced apoptosis. Accordingly, a useful T4SS interactor polypeptide includes an amino acid sequence at least about 45%, preferably 55%, 65%, 75%, 85%, 95%, or 99% identical to the amino acid sequence of SEQ ID NO:2 and retains the functional activity of the T4SS interactor polypeptide of SEQ ID NO:2. In other instances, the T4SS interactor polypeptide has an amino acid sequence 55%, 65%, 75%, 85%, 95%, or 98% identical to the T4SS interactor LRR domain. In one embodiment, the T4SS interactor polypeptide retains at least one functional activity of the T4SS interactor protein of SEQ ID NO:2.

To determine the percent identity of two amino acid sequences or of two nucleic acids, the sequences are aligned for optimal comparison purposes, wherein gaps are introduced in the sequences being compared. The amino acid residues at corresponding amino acid positions or nucleotides at corresponding nucleotide positions are then compared. When a position in a first sequence is occupied by the same amino acid residue or nucleotide as the corresponding position in a second sequence, then the molecules are identical at that position. The percent identity between the two sequences is a function of the number of identical positions shared by the sequences (e.g., % identity=# of identical positions/total # of positions ×100).

As described herein, the determination of percent homology between two sequences can be accomplished using a mathematical algorithm. Examples of a mathematical algorithm utilized for the comparison of two sequences is the algorithm of Karlin and Altschul (1993) Proc. Nat'l Acad. Sci. USA 90: 5873-5877 and the algorithm incorporated into the NBLAST and XBLAST programs of Altschul, et al. (1990) J. Mol. Biol. 215:403-410. Other examples of mathematical algorithms utilized for the comparison of sequences is the algorithm of Myers and Miller, CABIOS (1989) and the OrthoMCL for the identification of orthologs.

Native T4SS interactor polypeptides can be isolated from cells or tissue sources using the purification schemes described herein. The present invention also provides methods of producing T4SS interactor polypeptides using recombinant DNA techniques. Alternative to recombinant expression, a T4SS interactor protein or polypeptide can be synthesized chemically using standard peptide synthesis techniques.

The invention also provides T4SS interactor chimeric or fusion proteins. As used herein, a T4SS interactor “chimeric protein” or “fusion protein” comprises a T4SS interactor polypeptide fused in-frame to an additional component (a non-T4SS interactor polypeptide). Within a T4SS interactor fusion protein, the T4SS interactor polypeptide can correspond to all or a portion of a T4SS interactor protein, and preferably, retain at least one biologically active portion of a T4SS interactor protein. The additional component can be fused to the N-terminus or C-terminus of the T4SS interactor polypeptide. An example of a fusion protein is a GST-T4SS interactor fusion protein in which the T4SS interactor sequences are fused to the C-terminus of the GST sequences. Such fusion proteins can facilitate the purification of recombinant T4SS interactor. Another example of a fusion protein is a T4SS interactor-immunoglobulin fusion protein in which all or part of T4SS interactor is fused to sequences derived from a member of the immunoglobulin protein family. The T4SS interactor-immunoglobulin fusion proteins of the invention can be used as immunogens to produce anti-T4SS interactor antibodies in a subject, to purify T4SS interactor ligands and in screening assays to identify molecules which inhibit the interaction of T4SS interactor with a member of the T4SS transfer channel (e.g., VirB4, VirB11, VirB8).

A T4SS interactor chimeric or fusion protein of the invention can be produced by standard recombinant DNA techniques. For example, DNA fragments coding for the different polypeptide sequences are ligated together in-frame in accordance with conventional techniques (e.g., using blunt-ended or stagger-ended termini for ligation, restriction enzyme digestion, and enzymatic ligation). In another embodiment, conventional techniques such as an automated DNA synthesizer can be used. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A T4SS interactor-encoding nucleic acid can be cloned into such an expression vector such that the fusion moiety is linked in-frame to the T4SS interactor protein.

The present invention also pertains to variants of T4SS interactor proteins or polypeptides which function as either T4SS interactor agonists (mimetics) or as T4SS interactor antagonists. Variants of the T4SS interactor protein can be generated by mutagenesis (e.g., discrete point mutation or truncation of the T4SS interactor protein).

Variants of the T4SS interactor polypeptide which function as either T4SS interactor agonists or as T4SS interactor antagonists can be identified by screening combinatorial libraries of mutants, e.g., truncation mutants of the T4SS interactor polypeptide for T4SS interactor polypeptide agonist or antagonist activity. There are a variety of methods which can be used to produce libraries of potential T4SS interactor variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes provides, in one mixture, of all of the sequences encoding the desired set of potential T4SS interactor sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198: 056; Ike et al. (1983) Nucleic Acid Res. 11:477). Several techniques are known in the art for screening gene products of combinatorial libraries made by point mutations or truncation, and for screening cDNA libraries for gene products having a selected property (e.g., a biased library).

The present invention also relates to an antibody or antigen binding fragment thereof that specifically binds to a mammalian T4SS interactor polypeptide. In one embodiment, the antibody or antigen binding fragment thereof specifically binds to mammalian T4SS interactor polypeptide encoded by an isolated nucleic acid that encodes SEQ ID NO: 2. In another embodiment, the antibody or antigen binding fragment thereof specifically binds to mammalian T4SS interactor polypeptide comprising SEQ ID NO: 2. In another embodiment, the present invention is an antibody (e.g., polyclonal, monoclonal) or antigen binding fragment thereof that specifically binds to a Rickettsia sibirica rsib_orf.1266 polypeptide, wherein the Rickettsia sibirica rsib_orf.1266 polypeptide is encoded by an isolated nucleic acid that encodes SEQ ID NO: 2. An isolated T4SS interactor protein, or a portion or fragment thereof, can be used as an immunogen to generate antibodies that bind T4SS interactor using standard techniques for polyclonal and monoclonal antibody preparation. The full-length T4SS interactor polypeptide or antigenic peptide fragments of the T4SS interactor polypeptide can be used as immunogens. For example, an antigenic peptide of T4SS interactor can comprise at least about 10, 12, 15, 20, 25, 30, 35, 40, 45, 50, 60, 70, 80, 90, 100 120, 140 160, 180, 200, 220, or 240 amino acid residues of the amino acid sequence shown in SEQ ID NO:2 and encompass an epitope of T4SS interactor such that an antibody raised against the peptide forms a specific immune complex with the T4SS interactor polypeptide. Particular epitopes encompassed by the antigenic peptide are regions of T4SS interactor that are located on the surface of the protein, e.g., hydrophilic regions.

Generally, a suitable subject, (e.g., rabbit, goat, mouse, rat, hamster or other mammal) is immunized with a T4SS interactor immunogen to prepare antibodies or antigen binding fragments thereof that specifically bind T4SS interactor. The T4SS interactor immunogen can further include an adjuvant, such as Freund's complete or incomplete adjuvant, or similar immunostimulatory agent. Immunization of a suitable subject with an immunogenic T4SS interactor preparation induces a polyclonal anti-T4SS interactor antibody response.

A molecule which specifically binds to T4SS interactor is a molecule which binds T4SS interactor, but does not substantially bind other molecules in a sample, e.g., a biological sample, which contains T4SS interactor. As used herein “antibody” includes full length antibodies or immunoglobulin molecules and immunologically active portions of immunoglobulin molecules. Immunologically active portions of immunoglobulin molecules include, for example, F(ab) and F(ab′)₂ fragments which can be generated by treating the antibody with an enzyme such as pepsin. The term “antibody” also includes polyclonal and monoclonal antibodies that bind T4SS interactor.

Polyclonal anti-T4SS interactor antibodies can be prepared as described above by immunizing a suitable subject with a T4SS interactor immunogen. The antibody molecules directed against T4SS interactor can be isolated from the mammal (e.g., from the blood) and further purified by well-known techniques (e.g., protein A chromatography). In addition, antibody-producing cells can be obtained from the subject and used to prepare monoclonal antibodies by standard techniques, such as the hybridoma technique originally described by Kohler and Milstein (1975) Nature 256:495-497. The technology for producing various antibodies monoclonal antibody hybridomas is well known (see generally Current Protocols in Immunology (1994) Coligan et al. (eds.) John Wiley & Sons, Inc., New York, N.Y.). A monoclonal anti-T4SS interactor antibody can also be identified and isolated by screening a recombinant combinatorial immunoglobulin library (e.g., an antibody phage display library) with T4SS interactor to thereby isolate immunoglobulin library members that bind T4SS interactor. Kits for generating and screening phage display libraries are commercially available (e.g., the Pharmacia Recombinant Phage Antibody System, Catalog No. 27-9400-01; and the Stratagene SurfZAP.™. Phage Display Kit, Catalog No. 240612).

The term “antibody” also includes chimeric and humanized monoclonal antibodies, comprising both human and non-human portions, which can be made using standard recombinant DNA techniques. Such chimeric and humanized monoclonal antibodies can be produced by recombinant DNA techniques known in the art.

An anti-T4SS interactor antibody (e.g., monoclonal antibody) can be used to isolate T4SS interactor by standard techniques, such as affinity chromatography or immunoprecipitation. An anti-T4SS interactor antibody can facilitate the purification of natural T4SS interactor from cells, recombinantly produced T4SS interactor expressed in host cells and chemically synthesized T4SS interactor. Moreover, an anti-T4SS interactor antibody can be used to detect T4SS interactor protein in a sample (e.g., in a cellular lysate or cell supernatant) and also to evaluate the quantity and expression pattern of the T4SS interactor protein. Anti-T4SS interactor antibodies can be used diagnostically to monitor protein levels in tissue as part of a clinical testing procedure (e.g., to determine the efficacy of a given treatment regimen). A detectable substance or tag can be coupled to the antibody to facilitate detection. Examples of detectable substances include enzymes, prosthetic groups, fluorescent materials, luminescent materials, bioluminescent materials, and radioactive materials.

The present invention also provides expression constructs (expression vectors) containing a nucleic acid encoding a T4SS interactor polypeptide or a portion thereof. Examples of vectors include plasmids and viral vector (e.g., replication defective retroviruses, adenoviruses and adeno-associated viruses). In a particular embodiment, the invention is directed to expression constructs comprising SEQ ID NO:1. In another embodiment, SEQ ID NO: 1 in the expression construct is operably linked to a regulatory sequence.

The expression constructs of the invention comprise a T4SS interactor nucleic acid of the invention operably linked to one or more regulatory sequences. In one embodiment, the expression construct comprises SEQ ID NO: 1. The regulatory sequence is selected based on the vector and host cell used for expression of T4SS interactor. As used herein “operably linked” indicates that the T4SS interactor nucleic acid is linked to the regulatory sequence(s) in a manner which allows for expression of the nucleotide sequence (e.g., in an in vitro transcription/translation system or in a host cell when the vector is introduced into the host cell). As used herein, a “regulatory sequence” includes promoters, enhancers and other expression control elements such as polyadenylation signals which direct constitutive expression or tissue-specific expression of a nucleic acid. Such regulatory sequences are described, for example, in Goeddel; Gene Expression Technology: Methods in Enzymology 185, Academic Press, San Diego, Calif. (1990). The vector used in the present invention depends on several factors such as the choice of the host cell to be transformed, the level of expression of protein desired, etc. When introduced into a host cell the vectors of the invention can be used to produce T4SS interactor proteins or polypeptides, including fusion proteins or peptides, encoded by nucleic acids as described herein (e.g., T4SS interactor proteins, mutant forms of T4SS interactor, fusion proteins).

The vectors of the invention can be designed for expression of T4SS interactor in prokaryotic or eukaryotic cells, e.g., bacterial cells such as E. coli, insect cells (using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase. The vectors described herein can also comprise a nucleic acid molecule of the invention cloned into the vector in an antisense orientation.

Another aspect of the invention pertains to host cells into which an expression vector of the invention has been introduced (recombinant cells). In one embodiment, a host cell of the present invention comprises a nucleic acid molecule that encodes the amino acid sequence of SEQ ID NO: 2. The term “host cell” refers to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be a prokaryotic or eukaryotic cell. For example, T4SS interactor protein can be expressed in bacterial cells (e.g., E. coli), insect cells, yeast cells or mammalian cells (e.g., Chinese hamster ovary cells (CHO) or COS cells). Other suitable host cells are known to those skilled in the art.

Vector DNA can be introduced into prokaryotic or eukaryotic cells using a variety of art-recognized techniques for introducing foreign nucleic acid (e.g., DNA) into a host cell. For example, calcium phosphate or calcium chloride co-precipitation, DEAE-dextran-mediated transfection, lipofection, or electroporation can be used. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. Optionally, a selectable marker (e.g., resistance to antibiotics) can be introduced into the host cells along with the nucleic acid encoding T4SS interactor to identify and select cells that include the nucleic acid. Examples of selectable markers include G418, hygromycin and methotrexate. Nucleic acid encoding a selectable marker can be introduced into a host cell on the same vector as that encoding T4SS interactor or can be introduced on a separate vector.

A host cell of the invention, such as a prokaryotic or eukaryotic host cell in culture, can be used to produce (express) a T4SS interactor polypeptide (e.g., rsib_orf.1266). Accordingly, the invention further provides methods for producing a T4SS interactor polypeptide using the host cells of the invention. In one embodiment, the method comprises culturing the host cell comprising nucleic acid encoding a T4SS interactor polypeptide or portion thereof under conditions in which (e.g., in a suitable medium) T4SS interactor polypeptide is produced. In another embodiment, the method further comprises isolating T4SS interactor polypeptide from the medium or the host cell. In a particular embodiment, the host cells also provide for methods of producing a Rickettsia sibirica rsib_orf.1266 polypeptide comprising culturing a host cell described herein under conditions in which the Rickettsia sibirica rsib_orf.1266 polypeptide is produced. The method can further comprising isolating the Rickettsia sibirica rsib_orf.1266 polypeptide from the cell. Also encompassed by the invention is the isolated Rickettsia sibirica rsib_orf.1266 polypeptide produced by the method. The present invention also relates to the isolated T4SS interactor polypeptide.

The T4SS interactor nucleic acid molecules, T4SS interactor polypeptides, and anti-T4SS interactor antibodies (also referred to herein as “active compounds” ) of the invention can be incorporated into pharmaceutical compositions suitable for administration. Such compositions typically comprise the nucleic acid molecule, protein, or antibody and a pharmaceutically acceptable carrier. As used herein, a “pharmaceutically acceptable carrier” includes solvents, dispersion media, coatings, antibacterial and antifungal agents, isotonic and absorption delaying agents, and the like, compatible with pharmaceutical administration. The use of such media and agents for pharmaceutically active substances is well known in the art.

A pharmaceutical composition of the invention is formulated to be compatible with its intended route of administration. Examples of routes of administration include parenteral, e.g., intravenous, intradermal, subcutaneous, oral, intranasal, transdermal (topical), transmucosal, and rectal administration (e.g., suppositories). The pharmaceutical compositions of the present invention can also include a sterile diluent such as water for injection, saline solution, fixed oils, polyethylene glycols, glycerine, propylene glycol or other synthetic solvents; antibacterial agents; antioxidants; chelating agents; buffers and agents for the adjustment of tonicity. The can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline or phosphate buffered saline (PBS). The carrier can be a solvent or dispersion medium containing, for example, water, ethanol and polyol (e.g.,, glycerol, propylene glycol). In addition, a coating (e.g., lecithin) or a surfactant can be used. Antibacterial and antifungal agents, (e.g., thimerosal) can also be included. Moreover, sugars, polyalcohols and sodium chloride can be included in the pharmaceutical composition. An agent which delays absorption, for example, aluminum monostearate and gelatin can also be used.

Oral compositions can include an inert diluent or an edible carrier and can be in the form of capsules (e.g., gelatin), pills or tablets. The tablets, pills or capsules, can contain a binder, an excipient, a lubricant, a sweetening agent or a flavoring agent. For administration by inhalation, the compounds are delivered in the form of an aerosol spray from pressured container or dispenser which contains a suitable propellant, e.g., a gas such as carbon dioxide, or a nebulizer.

In one embodiment, the active compounds can be administered as a controlled release formulation, including implants and microencapsulated delivery systems (e.g., biodegradable, biocompatible polymers can be used). Methods for preparation of such formulations will be apparent to those skilled in the art. The materials can also be obtained commercially.

The dosage of the pharamceutical compositions of the invention depend on the unique characteristics of the active compound and the particular therapeutic effect to be achieved, and the limitations inherent in the art of compounding such an active compound for the treatment of individuals.

The pharmaceutical compositions can be included in a kit, container, pack, or dispenser together with instructions for administration.

The nucleic acid molecules, proteins, protein homologues, and antibodies described herein can be used in a variety of methods.

The isolated nucleic acid molecules of the invention can be used to express T4SS interactor protein (e.g., via a recombinant expression vector in a host cell), to detect T4SS interactor mRNA (e.g., in a biological sample) and to modulate T4SS interactor activity. In addition, the T4SS interactor proteins can be used to screen drugs or compounds which modulate the T4SS interactor activity or expression as well as to treat disorders associated with pathogens that utilize T4SS (e.g., a Spotted Fever Group pathogen such as R. sibirica). In addition, the anti-T4SS interactor antibodies of the invention can be used to detect and isolate T4SS interactor proteins and modulate T4SS interactor activity. This invention further pertains to novel agents identified by the above-described screening assays and their use for treatments as described herein.

Another aspect of the present invention relates to diagnostic assays for determining T4SS interactor polypeptide and/or nucleic acid expression as well as T4SS interactor activity, in the context of a biological sample (e.g., blood, serum, cells, tissue) to thereby determine whether an individual is afflicted with a pathogen that utilizes T4SS.

The present invention also pertains to a method for detecting the presence or absence of T4SS interactor in a sample (e.g., a biological sample) comprising contacting a sample with a compound or an agent capable of detecting T4SS interactor polypeptide or nucleic acid (e.g., mRNA, genomic DNA) that encodes T4SS interactor polypeptide such that the presence of T4SS interactor is detected in the sample. The method can further comprise obtaining the sample. In one embodiment, a labeled nucleic acid sequence (probe) capable of hybridizing to T4SS interactor mRNA or genomic DNA is used to detect T4SS interactor nucleic acid (e.g., mRNA or genomic DNA). The nucleic acid sequence can be, for example, a full-length T4SS interactor nucleic acid, such as the nucleic acid of SEQ ID NO: 1 or a portion thereof, such as an oligonucleotide of at least about 10, 20, 30, 50, 100, 350, 500, 600 or 700 nucleotides in length and sufficient to specifically hybridize under stringent conditions to T4SS interactor nucleic acid. Other suitable probes for use in the diagnostic assays of the invention are described herein.

For example, the present invention provides a method of identifying a nucleic acid that encodes a Rickettsia polypeptide in a sample (e.g., blood, lymph, urine, tissue) comprising contacting the sample with a complement of a nucleotide sequence comprising SEQ ID NO: 1 under conditions in which hybridization occurs between the complement and nucleic acid in the sample using high stringency conditions. The nucleic acid which hybridizes to the complement of the nucleotide sequence comprising SEQ ID NO: 1 under high stringency conditions is then identified. In one embodiment, the present invention relates to a method of identifying a nucleic acid that encodes a Rickettsia sibirica rsib_orf.1266 polypeptide in a sample comprising contacting the sample with a complement of a nucleotide sequence comprising SEQ ID NO: 1 under conditions in which hybridization occurs between the complement and nucleic acid in the sample using high stringency conditions.

In another embodiment, an antibody, such as an antibody with a detectable label, capable of binding to T4SS interactor protein or a characteristic portion thereof is used. Thus, the present invention also provides a method of identifying a T4SS interactor polypeptide in a sample comprising contacting the sample with an antibody or antigen binding fragment thereof that specifically binds to a T4SS interactor polypeptide wherein the T4SS interactor polypeptide is encoded by an isolated nucleic acid that encodes SEQ ID NO: 2. The polypeptide which specifically binds to the antibody is identified, thereby identifying a T4SS interactor polypeptide in a sample.

In a particular embodiment, the invention relates to a method of identifying a Rickettsia polypeptide in a sample comprising contacting the sample with an antibody or antigen binding fragment thereof that specifically binds to a Rickettsia sibirica rsib_orf.1266 polypeptide, wherein the Rickettsia sibirica rsib_orf.1266 polypeptide is encoded by an isolated nucleic acid that encodes SEQ ID NO: 2. The polypeptide which specifically binds to the antibody is then identified. In another embodiment, the invention relates to a method of identifying a Rickettsia sibirica rsib_orf.1266 polypeptide in a sample comprising contacting the sample with an antibody or antigen binding fragment thereof that specifically binds to a Rickettsia sibirica rsib_orf.1266 polypeptide, wherein the Rickettsia sibirica rsib_orf.1266 polypeptide is encoded by an isolated nucleic acid that encodes SEQ ID NO: 2.

Antibodies can be polyclonal, or more preferably, monoclonal. An intact antibody, or a fragment thereof (e.g., Fab or F(ab′)₂) can be used. Examples of detectable labels include a fluorescently labeled secondary antibody, and biotin such that it can be detected with fluorescently labeled streptavidin.

A “sample” includes biological samples such as tissues, cells and biological fluids of a subject which contain T4SS interactor protein molecules, mRNA molecules or genomic DNA molecules from the test subject. The detection method of the invention can be used to detect T4SS interactor mRNA, protein, or genomic DNA in a biological sample in vitro as well as in vivo. For example, in vitro techniques for detection of T4SS interactor mRNA include Northern hybridizations and in situ hybridizations. In vitro techniques for detection of T4SS interactor protein include enzyme linked immunosorbent assays (ELISAs), Western blots, immunoprecipitations and immunofluorescence. In vitro techniques for detection of T4SS interactor genomic DNA include Southern hybridizations. Furthermore, in vivo techniques for detection of T4SS interactor protein include introducing into a subject a labeled anti-T4SS interactor antibody, wherein the antibody is labeled with a radioactive marker whose presence and location in a subject can be detected by standard imaging techniques.

In another embodiment, the methods further involve obtaining a control sample from a control subject, contacting the control sample with a compound or agent capable of detecting T4SS interactor protein, mRNA, or genomic DNA, such that the presence of T4SS interactor protein, mRNA or genomic DNA is detected in the biological sample, and comparing the presence of T4SS interactor protein, mRNA or genomic DNA in the control sample with the presence of T4SS interactor protein, mRNA or genomic DNA in the test sample.

The invention also encompasses kits for detecting the presence of T4SS interactor in a sample. Such kits can be used to determine if a subject is suffering from or is at increased risk of developing a disorder associated with a pathogent that utilizes T4SS. For example, the kit can comprise a labeled compound or agent capable of detecting T4SS interactor protein or mRNA in a sample and means for determining the amount of T4SS interactor in the sample (e.g., an anti-T4SS interactor antibody or an oligonucleotide probe which binds to DNA encoding T4SS interactor such as SEQ ID NO:1). Kits may also include instruction for observing that the tested subject is suffering from or is at risk of developing a disorder associated with a pathogen that utilizes the T4SS.

For antibody-based kits, the kit may comprise, for example: (1) a first antibody (e.g., attached to a solid support) which binds to T4SS interactor polypeptide; and, optionally, (2) a second, different antibody which binds to T4SS interactor polypeptide or the first antibody and is conjugated to a detectable agent. For oligonucleotide-based kits, the kit may comprise, for example: (1) a oligonucleotide, e.g., a detectably labelled oligonucleotide, which hybridizes to a T4SS interactor nucleic acid sequence or (2) a pair of primers useful for amplifying a T4SS interactor nucleic acid molecule.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained along with instructions for observing whether the tested subject is suffering from or is at risk of developing a disorder associated with aberrant expression of T4SS interactor.

The invention provides a method (also referred to herein as a “screening assay”) for identifying agents that alter T4SS interactor expression and/or activity. For example, such agents (modulators) include candidate or test compounds or agents (e.g., peptides, peptidomimetics, small molecules such as small organic molecules or other drugs) which bind to a T4SS interactor polypeptide and/or inhibit or enhance (partially, completely) T4SS interactor expression or T4SS interactor activity. In one embodiment, the ability of an agent to alter T4SS interactor expression and/or activity is accomplished by determining the ability of the agent to alter the activity of (e.g., interaction of) T4SS interactor with a T4SS interactor target molecule (e.g., VirB4, VirB11, VirB8). As used herein, a “target molecule” is a molecule with which a T4SS interactor protein binds to or interacts with in nature. Thus, the present invention relates to a method of identifying an agent that alters interaction of a T4SS interactor protein with a target molecule comprising contacting a T4SS interactor protein having an amino acid sequence comprising SEQ ID NO: 2 with the target molecule under conditions in which the T4SS interactor protein interacts with the target molcecule, with an agent to be assessed. The extent to which T4SS interactor interacts with the target moclecule in the presence of the agent to be assessed is determined, wherein if the extent to which T4SS interactor interacts with the target molecule is altered in the presence of the agent compared to the extent to which T4SS interactor interacts with the target molecule in the absence of the agent, then the agent alters interaction of a mammalian T4SS interactor protein with the target molecule.

Thus, in particular embodiments, the present invention relates to a method of identifying an agent that alters (e.g., inhibits/enhances directly or indirectly) interaction of a polypeptide of a pathogen with a Type IV secretion system (T4SS) polypeptide, wherein the pathogen utilizes the T4SS (e.g., for infection) and the polypeptide has a leucine rich repeat domain, comprising contacting a Rickettsia sibirica rsib_orf.1266 polypeptide having an amino acid sequence comprising SEQ ID NO: 2 with the Type IV secretion system polypeptide under conditions in which the Rickettsia sibirica rsib_orf.1266 polypeptide interacts with the Type IV secretion system polypeptide, with an agent to be assessed. The extent to which Rickettsia sibirica rsib_orf.1266 polypeptide interacts with the Type IV secretion system polypeptide (e.g., VirD4, VirB11 and VirB8) in the presence of the agent to be assessed is then determined, wherein if the extent to which Rickettsia sibirica rsib_orf.1266 polypeptide interacts with the Type IV secretion system polypeptide is altered in the presence of the agent compared to the extent to which Rickettsia sibirica rsib_orf.1266 polypeptide interacts with the Type IV secretion system polypeptide in the absence of the agent, then the agent alters interaction of a polypeptide of a pathogen with a Type IV secretion system polypeptide.

In another embodiment, the invention relates to a method of identifying an agent that alters interaction of a Rickettsia polypeptide with a Type IV secretion system polypeptide comprising contacting a Rickettsia sibirica rsib_orf.1266 polypeptide having an amino acid sequence comprising SEQ ID NO: 2 with the Type IV secretion system polypeptide under conditions in which the Rickettsia sibirica rsib_orf.1266 polypeptide interacts with the Type IV secretion system polypeptide, with an agent to be assessed. The extent to which Rickettsia sibirica rsib_orf.1266 polypeptide interacts with the Type IV secretion system polypeptide in the presence of the agent to be assessed is determined, wherein if the extent to which Rickettsia sibirica rsib_orf.1266 polypeptide interacts with the Type IV secretion system polypeptide is altered in the presence of the agent compared to the extent to which Rickettsia sibirica rsib_orf.1266 polypeptide interacts with the Type IV secretion system polypeptide in the absence of the agent, then the agent alters interaction of a Rickettsia polypeptide with a Type IV secretion system polypeptide.

In yet another embodiment, the invention relates to a method of identifying an agent that alters interaction of a Rickettsia sibirica rsib_orf.1266 polypeptide with a Type IV secretion system polypeptide comprising contacting the Rickettsia sibirica rsib_orf.1266 polypeptide having an amino acid sequence comprising SEQ ID NO: 2 with the Type IV secretion system polypeptide under conditions in which the Rickettsia sibirica rsib_orf.1266 polypeptide interacts with the Type IV secretion system polypeptide, with an agent to be assessed. The extent to which Rickettsia sibirica rsib_orf.1266 polypeptide interacts with the Type IV secretion system polypeptide in the presence of the agent to be assessed, is then determined, wherein if the extent to which Rickettsia sibirica rsib_orf.1266 polypeptide interacts with the Type IV secretion system polypeptide is altered in the presence of the agent compared to the extent to which Rickettsia sibirica rsib_orf.1266 polypeptide interacts with the Type IV secretion system polypeptide in the absence of the agent, then the agent alters interaction of a Rickettsia sibirica rsib_orf.1266 polypeptide with the Type IV secretion system polypeptide.

Determining the ability of the T4SS interactor protein to bind to or interact with a T4SS interactor target molecule can be accomplished by methods which detect binding directly or indirectly. In one embodiment, determining the ability of the T4SS interactor protein to bind to or interact with a T4SS interactor target molecule can be accomplished by directly detecting the binding of T4SS interactor to the target molecule using, for example, one or more antibodies to detect T4SS interactor and/or its target molecule, or gel electrophoresis. In another embodiment, determining the ability of the T4SS interactor protein to bind to or interact with a T4SS interactor target molecule can be accomplished by determining the activity of T4SS interactor and/or the target molecule. For example, the activity of T4SS interactor or a T4SS interactor target molecule such as VirB4, can be determined by detecting interaction of T4SS interactor and VirB4, the ability of VirB4 to participate in the T4SS.

In other embodiments, the method comprises contacting a T4SS interactor protein or biologically active portion thereof with an agent and determining the ability of the agent to bind to the T4SS interactor protein or biologically active portion thereof. Binding of the test compound to the T4SS interactor protein can be determined either directly or indirectly. The assay can include contacting the T4SS interactor protein or biologically active portion thereof with a T4SS interactor target molecule which binds T4SS interactor (e.g., VirB11) to form an assay mixture; contacting the assay mixture with an agent; and determining the ability of the agent to interact with a T4SS interactor protein. In this embodiment, the ability of the agent to interact with a T4SS interactor protein comprises comparing the extent to which the agent binds to T4SS interactor or a biologically active portion thereof, to the extent to which the T4SS interactor target molecule binds to T4SS interactor or a biologically active portion thereof. If T4SS interactor preferentially binds the agent as compared to the T4SS interactor target molecule, then the agent alters T4SS interactor expression and/or activity.

In a particular embodiment, the invention relates to a method of identifying an agent that alters interaction of a polypeptide of a pathogen with a Type IV secretion system (T4SS) polypeptide, wherein the pathogen utilizes the T4SS and the polypeptide has a leucine rich repeat domain comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is then assessed, wherein if apoptosis of the cell is altered compared to the apoptosis of a control cell, then the agent alters interaction of a Rickettsia polypeptide with a Type IV secretion system polypeptide.

In another embodiment, the invention relates to a method of identifying an agent that alters interaction of a Rickettsia polypeptide with a Type IV secretion system polypeptide comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is then assessed, and if apoptosis of the cell is altered compared to the apoptosis of a control cell, then the agent alters interaction of a Rickettsia polypeptide with Type IV secretion system polypeptide.

In another embodiment, the invention relates to a method of identifying an agent that alters interaction of a Rickettsia sibirica rsib_orf.1266 polypeptide with a Type IV secretion system polypeptide comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is then assessed, and if apoptosis of the cell is altered compared to the apoptosis of a control cell, then the agent alters interaction of a Rickettsia sibirica rsib_orf.1266 polypeptide with Type IV secretion system polypeptide.

In the screening methods of the present invention, the T4SS interactor or its target molecule can be immobilized to facilitate separation of complexed from uncomplexed forms of one or both of the proteins, as well as to accommodate automation of the assay. Binding of an agent to T4SS interactor, or interaction of T4SS interactor with a target molecule in the presence and absence of an agent to be assessed, can be accomplished using, for example, microtitre plates, test tubes, and micro-centrifuge tubes. Examples of methods for immobilizing proteins on matrices include the use of glutathione-S-transferase/T4SS interactor fusion proteins or glutathione-S-transferase/target fusion proteins adsorbed onto glutathione sepharose beads (Sigma Chemical; St. Louis, Mo.) or glutathione derivatized microtitre plates and the use biotin and streptavidin conjugation. In another embodiment, modulators of T4SS interactor expression are identified in a method in which a cell is contacted with an agent and the expression of T4SS interactor mRNA or protein in the cell is determined. The level of expression of T4SS interactor mRNA or protein in the presence of the agent is compared to the level of expression of T4SS interactor mRNA or protein in the absence of the agent. The agent can then be identified as a modulator of T4SS interactor expression based on this comparison. For example, when expression of T4SS interactor mRNA or protein is greater in the presence of the agent than in its absence, the candidate compound is identified as a stimulator of T4SS interactor mRNA or protein expression. Alternatively, when expression of T4SS interactor mRNA or protein is less in the presence of the agent than in its absence, the candidate compound is identified as an inhibitor of T4SS interactor mRNA or protein expression. The level of T4SS interactor mRNA or protein expression in the cells can be determined by methods described herein for detecting T4SS interactor mRNA or protein.

The present invention also relates to a method of identifying an agent that inhibits (e.g., partially/completely; directly/indirectly) an interaction of a polypeptide of a pathogen with a Type IV secretion system (T4SS) polypeptide, wherein the pathogen utilizes the T4SS and the polypeptide has a leucine rich repeat domain comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is then determined, wherein an increase in apoptosis of the cell compared to apoptosis of a control cell indicates that the agent inhibits interaction of a polypeptide of a pathogen with a Type IV secretion system (T4SS) polypeptide.

In one embodiment, the present invention relates to a method of identifying an agent that inhibits an interaction of a Rickettsia polypeptide with a Type IV secretion system polypeptide comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib-orf.1266 polypeptide, when expressed, interacts with Type IV secretion system polypeptide in the cell, with an agent to be assessed. Whether apoptosis of the cell occurs is then determined, wherein an increase in apoptosis of the cell compared to apoptosis of a control cell indicates that the agent inhibits interaction of Rickettsia polypeptide with the Type IV secretion system polypeptide.

In another embodiment, the invention relates to a method of identifying an agent that inhibits an interaction of a Rickettsia sibirica rsib_orf.1266 polypeptide with a Type IV secretion system polypeptide comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed. Whether apoptosis of the cell occurs is assessed, wherein an increase in apoptosis of the cell compared to apoptosis of a control cell indicates that the agent inhibits interaction of Rickettsia sibirica rsib_orf.1266 polypeptide with the Type IV secretion system polypeptide.

The present invention also relates to a method of identifying an agent that enhances interaction of a polypeptide of a pathogen with a Type IV secretion system (T4SS) polypeptide, wherein the pathogen utilizes the T4SS and the polypeptide has a leucine rich repeat domain comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with the Type IV secretion system polypeptide in the cell, with an agent to be assessed. Whether apoptosis of the cell occurs is assessed, wherein a decrease in apoptosis of the cell compared to apoptosis of a control cell indicates that the agent enhances interaction of a polypeptide of a pathogen with a Type IV secretion system (T4SS) polypeptide.

The present invention also relates to a method of identifying an agent that enhances interaction of a Rickettsia polypeptide with a Type IV secretion system polypeptide comprising contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with the Type IV secretion system polypeptide in the cell, with an agent to be assessed. Whether apoptosis of the cell occurs is assessed, wherein a decrease in apoptosis of the cell compared to apoptosis of a control cell indicates that the agent enhances interaction of a Rickettsia polypeptide with Type IV secretion system polypeptide.

In a particular embodiment, the invention relates to a method of identifying an agent that enhances interaction of a Rickettsia sibirica rsib_orf.1266 polypeptide with a Type IV secretion system polypeptide comprising contacting a cell which comprises nucleic acid that encodes a Rickettsia sibirica rsib_orf1266 polypeptide having an amino acid sequence comprising SEQ ID NO: 2 wherein the Rickettsia sibirica rsib_orf.1266 polypeptide, when expressed, interacts with the Type IV secretion system polypeptide in the cell, with an agent to be assessed. Whether apoptosis of the cell occurs is assessed, wherein a decrease in apoptosis of the cell compared to apoptosis of a control cell indicates that the agent enhances interaction of a Rickettsia sibirica rsib_orf.1266 polypeptide with Type IV secretion system polypeptide.

The present invention also provides for prophylactic and therapeutic methods of treating a subject at risk of or susceptible to a disorder or having a disorder associated with a pathogen that utilizes the T4SS. In one aspect, the invention provides a method for preventing in a subject, a disease or condition associated with a pathogen that utilizes the T4SS, by administering to the subject an agent which alters T4SS interactor expression or at least one T4SS interactor activity. Subjects at risk for a disease which is caused or contributed to by a pathogen that utilizes the T4SS is identified by, for example, any of a combination of diagnostic or prognostic assays as described herein. Administration of a prophylactic agent can occur prior to the manifestation of symptoms characteristic of the T4SS interactor, such that a disease or disorder is prevented or, alternatively, delayed in its progression. Depending on the type of T4SS interactor, for example, a T4SS interactor agonist or T4SS interactor antagonist agent can be used for treating the subject. The appropriate agent can be determined based on screening assays described herein.

Another aspect of the invention pertains to methods of modulating T4SS interactor expression or activity for therapeutic purposes. The method of the invention involves contacting a cell with an agent that alters one or more of the activities of T4SS interactor polypeptide activity associated with the cell. An agent that alters T4SS interactor polypeptide activity can be an agent as described herein, such as a nucleic acid or a protein, a naturally-occurring cognate ligand of a T4SS interactor protein, a peptide, a T4SS interactor peptidomimetic, or other small molecule (e.g., small organic molecule). In one embodiment, the agent stimulates one or more of the biological activities of T4SS interactor protein. Examples of such stimulatory agents include active T4SS interactor protein and a nucleic acid molecule encoding T4SS interactor that has been introduced into the cell. In another embodiment, the agent inhibits one or more of the biological activities of T4SS interactor protein. Examples of such inhibitory agents include antisense or siRNA T4SS interactor nucleic acid molecules and anti-T4SS interactor antibodies. These methods can be performed in vitro (e.g., by culturing the cell with the agent) or, alternatively, in vivo (e.g, by administering the agent to a subject). As such, the present invention provides methods of treating an individual afflicted with a disease or disorder characterized by a pathogen that utilizes the T4SS. In one embodiment, the method involves administering an agent (e.g., an agent identified by a screening assay described herein), or combination of agents that modulates (e.g., upregulates or downregulates) T4SS interactor expression or activity.

In a particular embodiment, the present invention relates to a method of treating an infection by a pathogen in an individual, wherein the pathogen utilizes a Type IV secretion system (T4SS), comprising administering to the individual an agent that inhibits interaction of a Rickettsia sibirica rsib_orf.1266 polypeptide with a Type IV secretion system polypeptide. In one embodiment, the invention relates to a method of treating a Rickettsia infection (e.g., Rickettsia sibirica, Rickettsia prowazekii, Rickettsia conorii, Rickettsia rickettsii, Rickettsia typhi) in an individual comprising administering to the individual an agent that inhibits interaction of a Rickettsia sibirica rsib_orf.1266 polypeptide with a Type IV secretion system polypeptide.

In addition, all or a portion of the polypeptides, or the nucleotide sequences which encode the polypetides, described herein can be used as immunogens to elicit an immune response in a host (e.g., mammal, such as primate (e.g., human), canine, feline, bovine) to pathogens which utilize the T4SS (e.g., R. Sibirica). For example, an effective amount of all or a portion of a polypeptide described herein (e.g., rsib_orf.1266) can be administered to an individual. In one embodiment, the immunogen can be administered by in vivo expression of a polynucleotide encoding such into a host. The immunogen can be administered before (to prevent) or after (to treat) the effects of the pathogen. The immunogen can be administered to a host that either exhibits the disease state caused by the pathogen or does not yet exhibit the disease state. Thus, the immunogen can be administered to a host either before or after the disease state is manifested in the host, and can result in prevention, amelioration, elimination or delay in the onset of the disease state caused by the pathogen.

As described herein a nucleic acid sequence that encodes a polypeptide that interacts with a Type IV Secretion System (T4SS) has been identified using a functional shotgun sequencing method. Briefly, the method involves producing a protein interaction map of a genome of a cell (e.g., Rickettsia sibirica) comprising constructing a shotgun library of the cell's genome. Fragments from the shotgun library are then ligated into bacterial two hybrid bait vectors. Bait vectors which comprise open reading frame (ORF) fragments that are cloned in-frame in the bait vectors are determined, thereby producing bait peptides of interest. The bait peptides of interest are then screened against a genomic prey library of the cell's genome to identify prey peptides of interest that interact with the bait peptides of interest in the cell, thereby producing a protein interaction map of the genome of the cell. In a particular embodiment, the method involves producing a protein interaction map of a genome of a cell comprising constructing a shotgun library of the cell's genome. Fragments from the shotgun library are ligated into bacterial two hybrid bait vectors which express the peptide encoded by the fragment fused to a DNA binding protein. Bait vectors which comprise open reading frame (ORF) fragments that are cloned in-frame in the bait vectors are determined, thereby producing bait peptides of interest. Fragments from the shotgun library into bacterial two hybrid prey vectors which express the peptide encoded by the fragment fused to an activation domain, thereby producing a genomic prey library. The bait peptides of interest are then screened against the genomic prey library to identify prey peptides of interest that interact with the bait peptides of interest in the cell, thereby producing a protein interaction map of the genome of the cell.

In another particular embodiment, the bait peptides of interest are expressed as fusion proteins with bacteriophage λcI protein. In yet another embodiment, the prey peptides of interest are expressed as fusion proteins with RNA polymerase (e.g., the RNA polymerase α subunit).

The bait peptides of interest can be screened against the genomic prey library comprising introducing a vector comprising a bait peptide of interest and a vector comprising a prey peptide of interest into a bacterial cell, wherein the bacterial cell comprises a reporter gene operably linked to a regulatory sequence to which the DNA binding protein can bind, and wherein the reporter gene is expressed when the DNA binding protein is bound to the regulatory sequence and the activation domain is recruited to the regulatory sequence, thereby producing colonies. Colonies which express a peptide encoded by the reporter gene are then identified and nucleic acids or peptides expressed in the colonies identified are sequenced, thereby identifying a prey peptide of interest that interacts with a bait peptide of interest in the cell. The method can further comprise selecting fragments of the cell's genome for screening as bait peptide of interest.

### EXEMPLIFICATION

Methods

Modification of the Bacterial Two-Hybrid vectors

Original vectors from the system developed by Hochschild and colleagues'⁰ were modified as follows: pACλcI32, and pBRstar (Shaywitz, A. J., et al., Mol. Cell Biol., 20:9409-9422 (2000)) were modified by re-introducing the Not1 site followed by a BstXI restriction site, XhoI restriction site plus 3 frame stop codons. Constructs were renamed pBAIT and pPREY respectively. To verify that vector modifications did not alter functionality, Gal11P and Gal4 fragments (Dove, S. L., et al., Genes & Dev., 12:745-754 (1998)), were cloned into pBAIT and pPREY, screened and the capability to interact verified.

Functional Shotgun Sequencing of Rickettsia sibirica

Genomic DNA from R. sibirica was randomly sheared using nebulization. Fragments of 750 bp were gel purified, adapted with BstXI adaptors and ligated into pBAI.T. 27,314 sequences were attempted from this pBAIT library yielding 25,210 successful reads with average assembled length of 621 bp. Sequence coverage of the approximately 1.25 Mbp genome by pBAIT sequences was 12.5X. Coverage of the genome was checked for significant deviation from the expected mean and no regions of unusual over- or under- representation were found. To improve and verify the assembly, a fosmid library was generated by shearing genomic DNA to average size of 40 kb followed by cloning using fosmid packaging. Paired-end reads from 5,044 fosmids clones were attempted, yielding 8,322 successful reads. Sequence coverage of the genome by fosmid reads was 3.3X.

Assembly and Annotation

Assembly was conducted using the Paracel Genome Assembler™ (Paracel, Pasadena, Calif., USA) and ordered by paired end reads. Protein coding regions were initially determined by GeneMarkS (Besemer, J., et al., Nucleci Acids Res., 29:2607-2618 (2001))and assigned function based on BLASTP analysis using the GenBank NR database. Sequences front interactions were annotated using the COG database to create protein families (Table 4).

Selection of In-Frame Fragments and Creation of the ORF Fragment Library

pBAIT clones containing in-frame fragments of genes were determined by translation of nucleotide sequence oriented by the vector/insert junction. Translated fragments were then searched against the set of determined ORFs of R. sibirica for similarity. Clones determined to be in frame were re-arrayed to fresh plates creating a set of ORF fragments for screening. For baits, 17 overlapping peptide fragments were found spaiming; VirD4 (rsib_orf.311) a.a.. 2-591, VirBII (rsib_orf.312) a.a. 108-334, VirB10 (rsib_orf.313) a.a. 9-89 and a.a.125-483, VirB9 (rsib_orf.314) a.a. 80-157, VirB8′ (rsi.bᵤorf.315) a.a. 83-243, VirB7 (rsib_orf.316) a.a. 39-52, VirB8 (rsib_orf.317) a..a. 3-227.

Screening in the Bacterial Two-Hybrid System

For screening, the Bacteriomatch™ Reporter Strain (Stratagene, La Jolla, Calif., USA) was used. This strain harbors the reporter episome pFWO62SD+bla (Shaywitz, A. J., et al., Mol. Cell Biol., 20:9409-9422 (2000)) used in reporter strain US3F′3.1. For the screening shotgun library, adapted R. sibirica DNA from the shotgun sequencing project was mixed at a 1000:1 ratio with a control insert Gal11p, to serve as a downstream positive control, ligated into pPREY vector, transformed and approximately 6 million colonies were plated. After overnight growth the colonies were scraped and plasmid DNA extracted using standard methods. For the ORF library, all pBAITs determined through sequencing to contain in-frame fragments were arrayed, grown to stationary phase and plasmid DNA prepared. Inserts were excised and re-ligated into pPREY sites maintaining directionality. The ligation was transformed, plated and approximately 2 million colonies were scraped for DNA preparation. One hundred clones from both the ORF and shotgun library pPREY library were sequenced to insure the library was random. pBAIT DNA from clones containing peptide fragments of interest was prepared in 96-well plates using standard alkaline lysis methods. Each peptide of interest was transformed using 100 ul of cells, 50 ng of pBAIT, and 50 ng of either ORF library or shotgun library pPREY DNA. This yielded 650,000 dual transformants on average. Dual transformants were plated on 25 cm² plates containing LB agar supplemented with 25 ug/ml EPTG, 300 ug/ml Carbenicillin, 2 ug/ml Tetracycline, 50 ug/ml Kanamycin, and. 12.5 ug/ml Chloramphenicol. Small aliquots were also plated on media lacking Carbenicillin to determine total dual transformation numbers. At this level of dual transformation, the ORF fragment library was oversampled approximately 160 times. In the case of the shotgun library this was approximately 40×X coverage of the proteome. All colonies, or up to 400 colonies, growing after 16 hrs were picked for secondary screening on Agar plates containing all previous ingredients plus IPTG and Xgal. Colonies yielding blue color after overnight incubation were picked for sequencing.

Categorization and Validation of Interactions

Interactions were categorized as follows: observed once, were assigned score 1; more than once were assigned score 2; and more than once by different fragments were assigned score 3. This categorization represents the levels of validation of any given interaction. All screening against libraries was conducted in conjunction with a negative control pBAIT expressing the Lambda cl alone. Colonies from these screens were sequenced and one false positive, rsib_orf.1344, was identified. Both plasmids from 24 randomly selected interactors were prepared and re-transformed into the selection strain. Twenty-three of the original 24 clones revealed reconstituted interactions corresponding well with B-galactosidase activity. This suggests that approximately 4% of interactions may have occurred due to breakthrough of the reporter strain.

Results

Protein interaction maps can reveal novel pathways and functional complexes (Eisenberg, D., et al. Nature, 405:823-826 (2000)), allowing “guilt by association” annotation of uncharacterized proteins (Oliver, S., et al., Nature, 403:601-602 (2000)). As described herein, a bacterial two-hybrid system was coupled with a whole genome shotgun sequencing approach for microbial genome analysis, addressing a need for large-scale protein interaction analysis. The first large-scale proteomics study using this system, integrating de novo genome sequencing with functional interaction mapping and annotation in a high-throughput format, is described herein. The approach has been applied by shotgun sequencing the genome of Rickettsia sibirica strain 246, an obligate intracellular human pathogen among the Spotted Fever Group Rickettsiae. The bacteria invade endothelial cells and cause lysis after large amounts of progeny have accumulated. Little is known about specific rickettsial virulence factors and their mode of pathogenicity. Analysis of the combined genomic sequence and protein-protein interaction data for a set of virulence related Type IV Secretion System (T4SS) proteins revealed over 250 interactions and provides insight into the mechanism of Rickettsial pathogenicity including evidence of a novel transported host effector.

The utility of protein interaction maps is extensively documented and it is well appreciated that genome annotation could benefit from protein interaction data (Walhout, A. J., et al., Science, 287:116-122 (2000); Uetz, P., et al., Nature, 403:623-627 (2000); Ito, T., et al., Proc. Natl. Acad. Sci., USA, 98:4569-4574 (2001); Ito, T., et al., Proc. Natl. Acad. Sci., USA, 97:1143-1147 (2000)). Two hybrid projects require protein-coding regions be cloned in expression vectors as reagents for genetic screens (Reboul, J., et al., Nat. Genet. 34:35-41 (2003)). To obtain these, a shotgun strategy is preferable. This approach rapidly generates multiple overlapping fragments for any region. Use of fragment libraries in two-hybrid screens has been shown to reduce false-negatives (Rain, J. C., et al., Nature, 409:211-215 (2001); Ward, D. V., et al., Proc. Natl. Acad. Sci., USA, 99:11493-11500 (2002)). An additional benefit of this strategy is the ability to localize domains responsible for interactions, in both bait and prey constructs, similar in concept to deletion studies. To link genome sequencing and protein interaction mapping in a pipeline, a bacterial version of the Yeast Two-Hybrid (Y2H) system is well suited. Bacterial two-hybrid (B2H) systems have been developed and proven reliable for several selected gene products (Hu, J. C., et al., Methods, 20:80-94 (2000); Ladant, D., et al., Res. Microbiol., 151:711-720 (2000)), but to date B2H has not been applied to large scale protein interaction mapping as with the well established Y2H system (Uetz, P., et al., Nature, 403:623-627 (2000); Ito, T., et al., Proc. Natl. Acad. Sci., USA, 97:1143-1147 (2000); Rain, J. C., Nature, 409:211-215 (2001)).

The B2H system used in this study described herein was developed by Hochschild and colleagues (Dove, S. L., Nature, 386:627-630 (1997); Dove, S. L.,et al., Genes & Devel., 12:745-754 (1998); Dove, S. L., et al., J Bacteriol., 183:6413-6421 (2001); Shaywitz, A. J., et al., Mol. Cell Biol., 20:9409-9422 (2000)) and is similar in concept to the standard Y2H system. This method allows for random cloning of fragments because proteins are fused C-terminal to binding or activation domains. Briefly, a protein of interest (the bait) is fused to lambda cI, a DNA binding domain, which binds to a lambda operator sequence, OR2, placed upstream of a weak promoter. In addition, a second protein of interest (the prey) is fused to the RNA Polymerase alpha subunit, an activation domain, which is part of the RNAP holoenzyrne (FIG. 1a). If the two proteins of interest interact, RNAP is recruited to the weak promoter causing increased transcription of the downstream reporter genes, Beta-lactamase and Beta-galactosidase (FIG. 1b). Utilizing this system, a process termed “Functional Shotgun Sequencing” in which a shotgun library is constructed in the bait vector, followed by determination of open reading frame (ORF) fragments that are cloned in frame and can be used as baits, was developed (FIG. 2). Since fusion proteins are generated from standard backbone vectors and expressed in E. coli, sequencing of inserts to determine interacting proteins is greatly simplified.

The genome of R. sibirica 246 was subjected to functional shotgun sequencing, assembly, gene identification and automated annotation. Sequencing reads assembled into 1 supercontig consisting of 7 ordered and oriented contigs. A total of 1,234 putative genes were identified having an average coding length of 787 bp (Table 1), comprising 972,024 protein-coding bases, or 324,008 amino acids. As expected, the identified R. sibirica genes displayed a high degree of sequence conservation with genes of R. conorii and R. prowazekii whose genomes are completely sequenced. A total of 3,932 sequences, when translated in frame with lambda cI, revealed a cloned fragment from a R. sibirica ORF. The 3,932 in-frame clones spanned 599,602 amino acids or about 1.85×proteome redundancy. At this level of proteome coverage, predicted missing coverage will be P=e^{-1.85} or about 15.7% (Lander, E. S., et al., Genomics, 2:231-239 (1988)). Thus, approximately 85% of the proteome, or 1,040 proteins, should be represented in the identified clone set. In fact, in-frame fragments from 986 ORFs covering 278,832 unique amino acids were observed, corresponding to 86% of all amino acids being covered at least once. These numbers agree well with predicted coverage. From this set of clones, we were able to select clones spanning regions of interest for screening against either the sheared genomic prey library or the shuttled ORF fragment prey library.

The region of the genome including the virulence cluster VirD4-VirB8 was selected for further study. The proteins encoded by genes in this region are of interest because of their apparent role in virulence and their relationship to the Type IV Secretion System (T4SS) found in numerous pathogens. In some organisms, the T4SS has been shown responsible for delivery of effector molecules to cells of the eukaryotic host organism. Studies have been conducted on interactions among subunits of the T4SS complex in several microbes (Ward, D. V., et al., Proc. Natl. Acad. Sci., USA, 99:11493-11400 (2002); Ohashi, N., et al., Infect. Immun., 70:2128-2138 (2002); Das, A., et al., J. Bacteriol., 182:758-763 (2000); Christie, P. J., et al., Mol. Microbiol., 40:294-305 (2001)). While interactions among the subunits have been characterized, interactions between the T4SS complex and other proteins, such as secreted effectors, have not been well characterized. Given the presumed conservation of interactions between ortholog pairs in different species, termed interologs (Matthews, L. R., et al., Genome Res., 11:2120-2126 (2001)), interactions among the T4SS subunits similar to those identified in other organisms using the Y2H system were expected to be obtained. Seventeen in-frame fragments spanning portions of the VirD4-VirB8 region of the genome were selected for screening as baits against a sheared genomic prey library and a shuttled ORF fragment prey library. Screens against the ORF fragment library identified almost all interactions found using the shotgun library and determination of which approach to use should be set based on the scale of screening. Screens against the ORF fragment library produced fewer false positives (small non-genic peptides that appear to interact ubiquitously), and required less sequencing. However, the Shotgun prey library provided better resolution of the interaction domains.

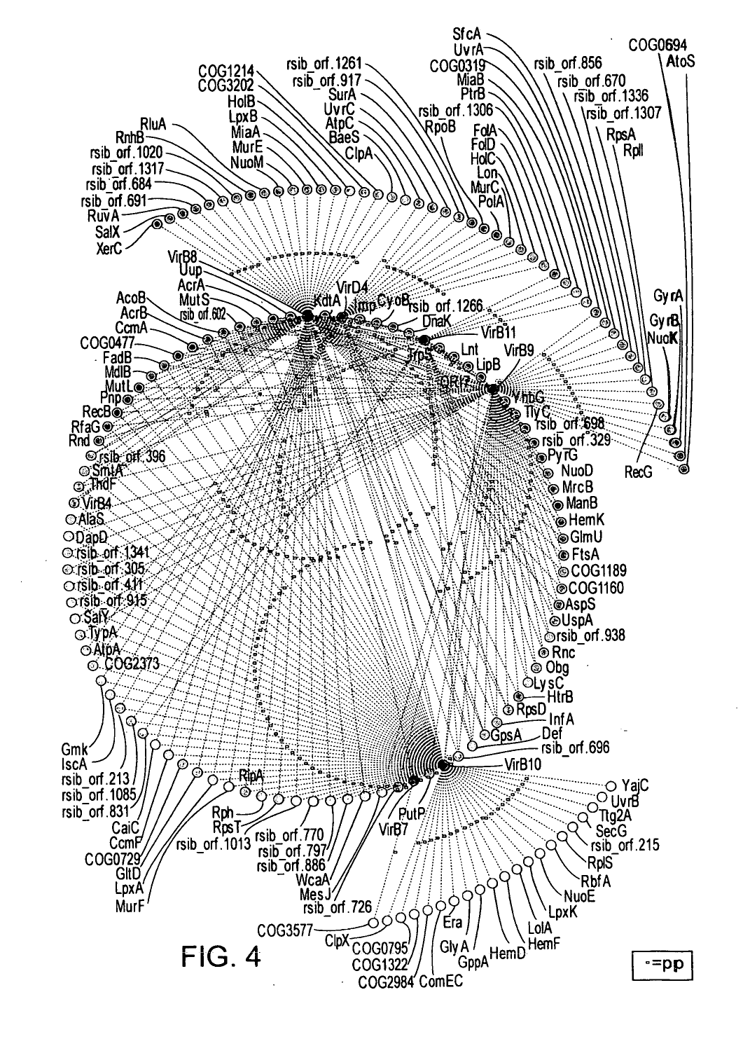

Screening yielded 285 distinct interactions between 155 proteins or protein families and the six T4SS subunits screened (FIG. 4 ). One hundred and sixty-two interactions fell into category 1 (observed once), 48 in category 2 (observed more than once) and 74 in category 3 (observed more than once using different fragments) (Supplementary Table 1). Forty percent of the interactions previously reported among T4SS subunits in other organisms using the Y2H were obtained using the B2H system (Table 3). This number is reasonable given the incomplete proteome coverage of the genome in the screen, and especially considering that previous investigations of interologs using the Y2H system reported between a 16% and 31% recapture rate (Matthews, L. R., et al., Genome Res., 11:2120-2126 (2001)). Interactions were found between the T4SS baits and lipopolysaccharide related proteins, hemolysins such as tlyC, protein export proteins, proteases, permeases, outer membrane proteins, ABC transporters, proteins of unknown function, and proteins of the T4SS complex among others. Of interest was an interaction between subunits of the T4SS and fadB. This gene was previously identified in a screen for genes expressed during intracellular infection. It was shown that fadB is activated in S. typhimurium specifically during intracellular infection (Mahan, M. J., et al., Proc. Natl.. Acad. Sci., USA, 92:669-673 (1995)). FadB is involved in Beta-oxidation of fatty acids that may help suppress host inflammatory response (Mahan, M. J., et al., Proc. Natl. Acad. Sci., USA, 92:669-673 (1995)). Observations of and interaction between the T4SS components and fadB strengthen the case for a role of this protein in pathogenicity.

Little is known of specific Rickettsial effectors that are secreted during infection (Clifton, D. R., et al., Proc. Natl. Acad. Sci., USA, 95:4646-4651 (1998)). One uncharacterized protein among the T4SS interactors in our screen, rsib_orf.1266, was found to contain a Leucine Rich Repeat (LRR) domain spanning the entire length of the protein, and most similar to the LRR in human NOD proteins (Inohara, N., et al., J. Biol. Chem., 274:214560-14567 (1999)). The protein from rsib_orf1266 interacts with VirD4, VirB11, and VirB8, all proposed members of the T4SS transfer channel (Christie, P. J., et al., Mol. Microbiol., 40:294-305 (2001)). Interaction with VirD4 is of significance because it is a coupling protein necessary for effector transport in A. tumefaciens and H. pylori (Christie, P. J., et al., Mol. Microbiol., 40:294-305 (2001)). LRR domains have been observed in effectors transported by the type III secretion system from a variety of intracellular plant and animal pathogens such as R. Solanaacearun (Salanoubat, M., et al., Nature, 415:497-502 (2002)) and Y pestis (Cornelis, G. R., et al., J. Cell Biol., 158:401-408). In addition, LRR domains have been found in the extracellular Internalin protein of microbes such L. monocytogenes and are involved in host cell internalization of the bacteria (Lecuit, M., et al., Infect. Immun., 65:5309-5319 (1997)). Of particular interest was the high similarity between rsib_orf. 1266 and the human NOD family (FIG. 3 ). NOD proteins are involved in bacterial component recognition in human cells through their LRR domain, and have been shown to activate NF-κB and Caspase activity, and subsequent apoptosis, by interacting with RICK (Inohara, N., et al., J. Biol. Chem., 276:2551-2554 (2001)). Crohn's disease, which is associated with mutations in the LRR of a NOD protein, results in cells incapable of bacterial component induced NF-κcB activation for apoptosis (Inohara, N., et al., Nat. Rev. Immunol., 3:371-382 (2003)). It has been shown that a fragment spanning the LRR domain of NOD1 alone was able to suppress RICK induced but not TNF alpha induced NF-κB activation (Inohara, N., et al., J. Biol. Chem., 274:14560-14567 (1999)). R. rickettsii, also a member of the Spotted Fever Group, has been shown to modulate NF-κB mediated host cell apoptosis during infection (Clifton, D. R., et al., Proc. Natl. Acad. Sci., USA, 95:4646-4651 (1998)). Despite evidence of their existence, however, no specific host apoptosis modulating effector molecules have been identified in the Rickettsiae. It is proposed herein that rsib_orf.1266, containing an LRR domain, is likely an effector transported by the T4SS, and also that rsib_orf.1266 likely acts as either an internalin, or as a “sink” for bacterial cell wall components, such as LPS and/or peptidoglycan, released during host cell infection. Binding of bacterial components by rsib_orf.1266 would act as a dominant negative NOD mutant disallowing activation of the NOD proteins and subsequent caspase induced apoptosis. This model host molecule mimicry allows for the observations that Rickettsiae activate NF-κB because TNF-alpha induced NF-κB activation could still proceed (Inohara, N., et al., J. Biol. Chem., 274:14560-14567 (1999)). A similar protein to Rsib_orf.1266 was found in R. conorii, but not in R. prowazekii, a Typhus Group Rickettsia, suggesting rsib_orf.1266 is a Spotted Fever Group specific effector.

By implementing a large-scale bacterial two-hybrid system, it has been demonstrated that it is possible to couple whole genome sequencing and protein interaction mapping in a standard sequencing pipeline. This approach and data will help in development of new drug targets by providing information on genes that are critical to the pathogenicity, maintenance, and spread of microbes.

The R. sibirica assembled and annotated whole genome shotgun sequence has been deposited in GenBank under accession number AABWO01000001

TABLE 1
Comparison of Spotted Fever Group Rickettsiae genomes

| | R. sibirica | R. conorii |
|---|---|---|
| Protein-Coding Regions | 1234 | 1373 |
| Average protein-coding | 787 | 746 |
| gene length (bp) | | |
| % coding | 77.7 | 80.8 |
| % G + C | 32.9 | 32.9 |
| Genome Size (bp) | 1,250,021 | 1,268,755 |

TABLE 2
T4SS Interactions

| Both | Previous Study | This Study |
|---|---|---|
| VirB9—VirB9 | VirB4-VirB11 | VirB10—VirB10 |
| VirB9-VirB10 | VirB10-VirB11 | VirB10-VirD4 |
| VirB9-VirB11 | VirB11-VirB8 | VirB10-VirB7 |
| VirB4-VirB10 | VirB7-VirB9 | VirB8-VirB7 |
| VirB4-VirB8 | VirB8—VirB8 | VirD4-VirB4 |
| | VirB8-VirB10 | |
| | VirB9-VirB8 | |

Column A contains interactions between the T4SS subunits that were captured in a study of A. tumefaciens T4SS interactions (Ward, D. V, et al., Proc. Natl. Acad. Sci., USA, 99:11493-11500 (2002) and in the study described herein.
Column B contains interactions obtained in the previous study and not captured in the study described herein.

Column C contains interactions among T4SS obtained in the study described herein but not in the previous study.

TABLE 3

| | | | validation_category |
|---|---|---|---|
| | | | 3 = mult_frag |
| bait | gene | description | 2 = mult_obs |
| VirB11 | AcoB | Pyruvate/2-oxoglutarate dehydrogenase | 1 |
| | | complex, dehydrogenase (E1) component, | |
| | | eukaryotic type, beta subunit | |
| VirB8 | AcoB | Pyruvate/2-oxoglutarate dehydrogenase | 1 |
| | | complex, dehydrogenase (E1) component, | |
| | | eukaryotic type, beta subunit | |
| VirB10 | AcoB | Pyruvate/2-oxoglutarate dehydrogenase | 3 |
| | | complex, dehydrogenase (E1) component, | |
| | | eukaryotic type, beta subunit | |
| VirB10 | AcrA | Membrane-fusion protein | 1 |
| VirB8 | AcrA | Membrane-fusion protein | 1 |
| VirB9 | AcrA | Membrane-fusion protein | 1 |
| VirD4 | AcrA | Membrane-fusion protein | 1 |
| VirB8 | AcrB | Cation/multidrug efflux pump | 1 |
| VirB10 | AcrB | Cation/multidrug efflux pump | 3 |
| VirB9 | AcrB | Cation/multidrug efflux pump | 3 |
| VirB10 | AlaS | Alanyl-tRNA synthetase | 2 |
| VirB9 | AlaS | Alanyl-tRNA synthetase | 2 |
| VirB8 | AspS | Aspartyl-tRNA synthetase | 1 |
| VirB9 | AspS | Aspartyl-tRNA synthetase | 1 |
| VirB9 | AtoS | FOG: PAS/PAC domain | 2 |
| VirB10 | AtpA | F0F1-type ATP synthase, alpha subunit | 1 |
| VirD4 | AtpA | F0F1-type ATP synthase, alpha subunit | 3 |
| VirB8 | AtpC | F0F1-type ATP synthase, epsilon subunit | 2 |
| | | (mitochondrial delta subunit) | |
| VirB8 | BaeS | Signal transduction histidine kinase | 2 |
| VirB8 | CaiC | Acyl-CoA synthetases (AMP-forming)/AMP-acid | 1 |
| | | ligases II | |
| VirB10 | CaiC | Acyl-CoA synthetases (AMP-forming)/AMP-acid | 2 |
| | | ligases II | |
| VirB10 | CcmA | ABC-type multidrug transport system, ATPase | 1 |
| | | component | |
| VirB8 | CcmA | ABC-type multidrug transport system, ATPase | 1 |
| | | component | |
| VirB9 | CcmA | ABC-type multidrug transport system, ATPase | 1 |
| | | component | |
| VirB10 | CcmF | Cytochrome c biogenesis factor | 1 |
| VirB8 | CcmF | Cytochrome c biogenesis factor | 2 |
| VirB8 | ClpA | ATPases with chaperone activity, ATP-binding | 1 |
| | | subunit | |
| VirB10 | ClpX | ATP-dependent protease Clp, ATPase subunit | 1 |
| VirB11 | COG0319 | Predicted metal-dependent hydrolase | 3 |
| VirB8 | COG0477 | Permeases of the major facilitator superfamily | 1 |
| VirB10 | COG0477 | Permeases of the major facilitator superfamily | 2 |
| VirB11 | COG0477 | Permeases of the major facilitator superfamily | 3 |
| VirB9 | COG0694 | Thioredoxin-like proteins and domains | 3 |
| VirB8 | COG0729 | Outer membrane protein | 1 |
| VirB10 | COG0729 | Outer membrane protein | 2 |
| VirB10 | COG0795 | Predicted permeases | 3 |
| VirB8 | COG1160 | Predicted GTPases | 1 |
| VirB9 | COG1160 | Predicted GTPases | 1 |
| VirB9 | COG1189 | Predicted rRNA methylase | 2 |
| VirB8 | COG1189 | Predicted rRNA methylase | 3 |
| VirB8 | COG1214 | Inactive homolog of metal-dependent proteases, | 2 |
| | | putative molecular chaperone | |
| VirB10 | COG1322 | Uncharacterized protein conserved in bacteria | 3 |
| VirB10 | COG2373 | Large extracellular alpha-helical protein | 1 |
| VirD4 | COG2373 | Large extracellular alpha-helical protein | 1 |
| VirB10 | COG2984 | ABC-type uncharacterized transport system, | 3 |
| | | periplasmic component | |
| VirB8 | COG3202 | ATP/ADP translocase | 2 |
| VirB7 | COG3577 | Predicted aspartyl protease | 1 |
| VirB10 | ComEC | Predicted hydrolase (metallo-beta-lactamase | 1 |
| | | superfamily) | |
| VirB8 | CyoB | Heme/copper-type cytochrome/quinol oxidases, | 1 |
| | | subunit 1 | |
| VirD4 | CyoB | Heme/copper-type cytochrome/quinol oxidases, | 1 |
| | | subunit 1 | |
| VirB10 | DapD | Tetrahydrodipicolinate N-succinyltransferase | 1 |
| VirB9 | DapD | Tetrahydrodipicolinate N-succinyltransferase | 1 |
| VirB11 | Def | N-formylmethionyl-tRNA deformylase | 1 |
| VirB8 | Def | N-formylmethionyl-tRNA deformylase | 1 |
| VirD4 | Def | N-formylmethionyl-tRNA deformylase | 1 |
| VirB9 | Def | N-formylmethionyl-tRNA deformylase | 3 |
| VirB11 | DnaK | Molecular chaperone | 2 |
| VirB8 | DnaK | Molecular chaperone | 2 |
| VirB9 | DnaK | Molecular chaperone | 3 |
| VirD4 | DnaK | Molecular chaperone | 3 |
| VirB10 | Era | GTPase | 2 |
| VirB10 | FadB | 3-hydroxyacyl-CoA dehydrogenase | 2 |
| VirB8 | FadB | 3-hydroxyacyl-CoA dehydrogenase | 2 |
| VirB9 | FadB | 3-hydroxyacyl-CoA dehydrogenase | 3 |
| VirD4 | FolA | Dihydrofolate reductase | 1 |
| VirD4 | FolD | 5,10-methylene-tetrahydrofolate | 1 |
| | | dehydrogenase/Methenyl tetrahydrofolate | |
| | | cyclohydrolase | |
| VirB9 | FtsA | Actin-like ATPase involved in cell division | 1 |
| VirB8 | FtsA | Actin-like ATPase involved in cell division | 2 |
| VirB8 | GlmU | N-acetylglucosamine-1-phosphate | 1 |
| | | uridyltransferase (contains nucleotidyltransferase | |
| | | and I-patch acetyltransferase domains) | |
| VirB9 | GlmU | N-acetylglucosamine-1-phosphate | 1 |
| | | uridyltransferase (contains nucleotidyltransferase | |
| | | and I-patch acetyltransferase domains) | |
| VirB8 | GltD | NADPH-dependent glutamate synthase beta | 1 |
| | | chain and related oxidoreductases | |
| VirB10 | GltD | NADPH-dependent glutamate synthase beta | 3 |
| | | chain and related oxidoreductases | |
| VirB10 | GlyA | Glycine/serine hydroxymethyltransferase | 3 |
| VirB10 | Gmk | Guanylate kinase | 1 |
| VirD4 | Gmk | Guanylate kinase | 1 |
| VirB10 | GppA | Exopolyphosphatase | 3 |
| VirD4 | GpsA | Glycerol-3-phosphate dehydrogenase | 1 |
| VirB8 | GpsA | Glycerol-3-phosphate dehydrogenase | 3 |
| VirB9 | GpsA | Glycerol-3-phosphate dehydrogenase | 3 |
| VirB9 | GyrA | Type IIA topoisomerase (DNA gyrase/topo II, | 3 |
| | | topoisomerase IV), A subunit | |
| VirB9 | GyrB | Type IIA topoisomerase (DNA gyrase/topo II, | 1 |
| | | topoisomerase IV), B subunit | |
| VirB10 | HemD | Uroporphyrinogen-III synthase | 1 |
| VirB10 | HemF | Coproporphyrinogen III oxidase | 3 |
| VirB8 | HemK | Methylase of polypeptide chain release factors | 1 |
| VirB9 | HemK | Methylase of polypeptide chain release factors | 2 |
| VirB8 | HolB | ATPase involved in DNA replication | 1 |
| VirD4 | HolC | DNA polymerase III, chi subunit | 3 |
| VirB9 | HtrB | Lauroyl/myristoyl acyltransferase | 3 |
| VirD4 | HtrB | Lauroyl/myristoyl acyltransferase | 3 |
| VirB8 | Imp | TRAP-type uncharacterized transport system, | 1 |
| | | periplasmic component | |
| VirD4 | Imp | TRAP-type uncharacterized transport system, | 1 |
| | | periplasmic component | |
| VirB11 | InfA | Translation initiation factor 1 (IF-1) | 1 |
| VirB8 | InfA | Translation initiation factor 1 (IF-1) | 1 |
| VirB9 | InfA | Translation initiation factor 1 (IF-1) | 3 |
| VirD4 | IscA | Uncharacterized conserved protein | 1 |
| VirB10 | IscA | Uncharacterized conserved protein | 2 |
| VirD4 | KdtA | 3-deoxy-D-manno-octulosonic-acid transferase | 1 |
| VirB10 | KdtA | 3-deoxy-D-manno-octulosonic-acid transferase | 2 |
| VirB9 | KdtA | 3-deoxy-D-manno-octulosonic-acid transferase | 2 |
| VirB8 | KdtA | 3-deoxy-D-manno-octulosonic-acid transferase | 3 |
| VirB11 | LipB | Lipoate-protein ligase B | 1 |
| VirB8 | LipB | Lipoate-protein ligase B | 1 |
| VirB11 | Lnt | Apolipoprotein N-acyltransferase | 1 |
| VirB8 | Lnt | Apolipoprotein N-acyltransferase | 1 |
| VirB10 | LolA | Outer membrane lipoprotein-sorting protein | 1 |
| VirD4 | Lon | ATP-dependent Lon protease, bacterial type | 3 |
| VirB10 | LpxA | Acyl-[acyl carrier protein]--UDP-N- | 1 |
| | | acetylglucosamine O-acyltransferase | |
| VirB8 | LpxA | Acyl-[acyl carrier protein]--UDP-N- | 1 |
| | | acetylglucosamine O-acyltransferase | |
| VirB8 | LpxB | Lipid A disaccharide synthetase | 1 |
| VirB10 | LpxK | Tetraacyldisaccharide-1-P 4′-kinase | 3 |
| VirB11 | LysC | Aspartokinases | 1 |
| VirB9 | LysC | Aspartokinases | 1 |
| VirB8 | ManB | Phosphomannomutase | 2 |
| VirB9 | ManB | Phosphomannomutase | 2 |
| VirD4 | MdlB | ABC-type multidrug transport system, ATPase | 2 |
| | | and permease components | |
| VirB10 | MdlB | ABC-type multidrug transport system, ATPase | 3 |
| | | and permease components | |
| VirB8 | MdlB | ABC-type multidrug transport system, ATPase | 3 |
| | | and permease components | |
| VirB7 | MesJ | Predicted ATPase of the PP-loop superfamily | 1 |
| | | implicated in cell cycle control | |
| VirB9 | MesJ | Predicted ATPase of the PP-loop superfamily | 1 |
| | | implicated in cell cycle control | |
| VirB10 | MesJ | Predicted ATPase of the PP-loop superfamily | 3 |
| | | implicated in cell cycle control | |
| VirB8 | MiaA | tRNA delta(2)-isopentenylpyrophosphate | 3 |
| | | transferase | |
| VirB11 | MiaB | 2-methylthioadenine synthetase | 1 |
| VirB8 | MrcB | Membrane carboxypeptidase (penicillin-binding | 1 |
| | | protein) | |
| VirB9 | MrcB | Membrane carboxypeptidase (penicillin-binding | 1 |
| | | protein) | |
| VirD4 | MurC | UDP-N-acetylmuramate-alanine ligase | 1 |
| VirB8 | MurE | UDP-N-acetylmuramyl tripeptide synthase | 3 |
| VirB10 | MurF | UDP-N-acetylmuramyl pentapeptide synthase | 1 |
| VirB8 | MurF | UDP-N-acetylmuramyl pentapeptide synthase | 1 |
| VirB10 | MutL | DNA mismatch repair enzyme (predicted | 1 |
| | | ATPase) | |
| VirB8 | MutL | DNA mismatch repair enzyme (predicted | 1 |
| | | ATPase) | |
| VirB9 | MutL | DNA mismatch repair enzyme (predicted | 1 |
| | | ATPase) | |
| VirB10 | MutS | Mismatch repair ATPase (MutS family) | 1 |
| VirB9 | MutS | Mismatch repair ATPase (MutS family) | 3 |
| VirD4 | MutS | Mismatch repair ATPase (MutS family) | 3 |
| VirB8 | NuoD | NADH: ubiquinone oxidoreductase 49 kD subunit 7 | 1 |
| VirB9 | NuoD | NADH: ubiquinone oxidoreductase 49 kD subunit 7 | 3 |
| VirB10 | NuoE | NADH: ubiquinone oxidoreductase 24 kD subunit | 1 |
| VirB9 | NuoK | NADH: ubiquinone oxidoreductase subunit 11 or | 1 |
| | | 4 L (chain K) | |
| VirB8 | NuoM | NADH: ubiquinone oxidoreductase subunit 4 | 1 |
| | | (chain M) | |
| VirB11 | Obg | Predicted GTPase | 1 |
| VirB9 | Obg | Predicted GTPase | 1 |
| VirB10 | Pnp | Polyribonucleotide nucleotidyltransferase | 1 |
| | | (polynucleotide phosphorylase) | |
| VirB9 | Pnp | Polyribonucleotide nucleotidyltransferase | 1 |
| | | (polynucleotide phosphorylase) | |
| VirB8 | Pnp | Polyribonucleotide nucleotidyltransferase | 2 |
| | | (polynucleotide phosphorylase) | |
| VirD4 | PolA | DNA polymerase I - 3′-5′ exonuclease and | 3 |
| | | polymerase domains | |
| VirB11 | PtrB | Protease II | 1 |
| VirB10 | PutP | Na+/proline symporter | 1 |
| VirB7 | PutP | Na+/proline symporter | 1 |
| VirB9 | PutP | Na+/proline symporter | 1 |
| VirB9 | PyrG | CTP synthase (UTP-ammonia lyase) | 2 |
| VirB8 | PyrG | CTP synthase (UTP-ammonia lyase) | 3 |
| VirB10 | QRI7 | Metal-dependent proteases with possible | 1 |
| | | chaperone activity | |
| VirB11 | QRI7 | Metal-dependent proteases with possible | 1 |
| | | chaperone activity | |
| VirB8 | QRI7 | Metal-dependent proteases with possible | 1 |
| | | chaperone activity | |
| VirB9 | QRI7 | Metal-dependent proteases with possible | 1 |
| | | chaperone activity | |
| VirB10 | RbfA | O-antigen export system permease protein RfbA | 1 |
| VirB9 | RecB | ATP-dependent exoDNAse (exonuclease V) beta | 1 |
| | | subunit (contains helicase and exonuclease | |
| | | domains) | |
| VirB8 | RecB | ATP-dependent exoDNAse (exonuclease V) beta | 2 |
| | | subunit (contains helicase and exonuclease | |
| | | domains) | |
| VirB10 | RecB | ATP-dependent exoDNAse (exonuclease V) beta | 3 |
| | | subunit (contains helicase and exonuclease | |
| | | domains) | |
| VirB9 | RecG | RecG-like helicase | 1 |
| VirB10 | RfaG | Glycosyltransferase | 1 |
| VirB8 | RfaG | Glycosyltransferase | 2 |
| VirB9 | RfaG | Glycosyltransferase | 3 |
| VirB10 | RlpA | Lipoproteins | 1 |
| VirB8 | RlpA | Lipoproteins | 2 |
| VirB8 | RluA | Pseudouridylate synthases, 23S RNA-specific | 3 |
| VirB11 | Rnc | dsRNA-specific ribonuclease | 1 |
| VirB9 | Rnc | dsRNA-specific ribonuclease | 1 |
| VirB10 | Rnd | Ribonuclease D | 1 |
| VirB11 | Rnd | Ribonuclease D | 1 |
| VirB8 | Rnd | Ribonuclease D | 1 |
| VirB8 | RnhB | Ribonuclease HII | 1 |
| VirB10 | Rph | RNase PH | 1 |
| VirB8 | Rph | RNase PH | 1 |
| VirB9 | Rpll | Ribosomal protein L9 | 3 |
| VirB10 | RplS | Ribosomal protein L19 | 3 |
| VirD4 | RpoB | DNA-directed RNA polymerase, beta subunit/140 | 1 |
| | | kD subunit | |
| VirB9 | RpsA | Ribosomal protein S1 | 3 |
| VirB8 | RpsD | Ribosomal protein S4 and related proteins | 1 |
| VirB9 | RpsD | Ribosomal protein S4 and related proteins | 1 |
| VirD4 | RpsD | Ribosomal protein S4 and related proteins | 1 |
| VirB10 | RpsT | Ribosomal protein S20 | 1 |
| VirB8 | RpsT | Ribosomal protein S20 | 1 |
| VirB10 | rsib_orf.1013 | outer membrane protein B (cell surface antigen | 1 |
| | | sca5) | |
| VirB8 | rsib_orf.1013 | outer membrane protein B (cell surface antigen | 1 |
| | | sca5) | |
| VirB8 | rsib_orf.1020 | unknown | 3 |
| VirB10 | rsib_orf.1085 | unknown | 1 |
| VirB11 | rsib_orf.1085 | unknown | 1 |
| VirD4 | rsib_orf.1261 | similarity to 3-hydroxyacyl-CoA dehydrogenase | 1 |
| | | (FadB) | |
| VirB11 | rsib_orf.1266 | unknown | 1 |
| VirD4 | rsib_orf.1266 | unknown | 2 |
| VirB8 | rsib_orf.1266 | unknown | 3 |
| VirB11 | rsib_orf.1306 | unknown | 3 |
| VirB9 | rsib_orf.1307 | unknown | 3 |
| VirB8 | rsib_orf.1317 | unknown | 2 |
| VirB10 | rsib_orf.1341 | unknown | 1 |
| VirB9 | rsib_orf.1341 | unknown | 1 |
| VirB9 | rsib_orf.1366 | unknown | 2 |
| VirB10 | rsib_orf.213 | unknown | 1 |
| VirD4 | rsib_orf.213 | unknown | 1 |
| VirB10 | rsib_orf.215 | unknown | 3 |
| VirB9 | rsib_orf.305 | unknown | 1 |
| VirB10 | rsib_orf.305 | unknown | 3 |
| VirB8 | rsib_orf.329 | unknown | 1 |
| VirB9 | rsib_orf.329 | unknown | 2 |
| VirB10 | rsib_orf.396 | unknown | 3 |
| VirB8 | rsib_orf.396 | unknown | 3 |
| VirD4 | rsib_orf.396 | unknown | 3 |
| VirB10 | rsib_orf.411 | unknown | 1 |
| VirB9 | rsib_orf.411 | unknown | 1 |
| VirB11 | rsib_orf.602 | 190 kD antigen precursor | 1 |
| VirD4 | rsib_orf.602 | 190 kD antigen precursor | 1 |
| VirB10 | rsib_orf.602 | 190 kD antigen precursor | 3 |
| VirB9 | rsib_orf.670 | unknown - Colicin V domain | 3 |
| VirB8 | rsib_orf.684 | unknown | 3 |
| VirB8 | rsib_orf.691 | unknown | 2 |
| VirB10 | rsib_orf.696 | unknown | 1 |
| VirB11 | rsib_orf.696 | unknown | 2 |
| VirB8 | rsib_orf.696 | unknown | 2 |
| VirB9 | rsib_orf.696 | unknown | 2 |
| VirB9 | rsib_orf.698 | cell surface antigen | 1 |
| VirB8 | rsib_orf.698 | cell surface antigen | 3 |
| VirB7 | rsib_orf.726 | similarity to O-linked GlcNAc transferase | 1 |
| VirB9 | rsib_orf.726 | similarity to O-linked GlcNAc transferase | 1 |
| VirB10 | rsib_orf.770 | unknown | 1 |
| VirB8 | rsib_orf.770 | unknown | 1 |
| VirB10 | rsib_orf.797 | unknown | 1 |
| VirB8 | rsib_orf.797 | unknown | 3 |
| VirB10 | rsib_orf.831 | unknown | 1 |
| VirB11 | rsib_orf.831 | unknown | 1 |
| VirB9 | rsib_orf.856 | unknown | 2 |
| VirB8 | rsib_orf.886 | unknown | 1 |
| VirB10 | rsib_orf.886 | unknown | 3 |
| VirB10 | rsib_orf.915 | unknown | 1 |
| VirB9 | rsib_orf.915 | unknown | 1 |
| VirD4 | rsib_orf.917 | unknown | 2 |
| VirB11 | rsib_orf.938 | unknown | 1 |
| VirB9 | rsib_orf.938 | unknown | 1 |
| VirB8 | RuvA | Holliday junction resolvasome, DNA-binding | 2 |
| | | subunit | |
| VirB8 | SalX | ABC-type antimicrobial peptide transport system, | 1 |
| | | ATPase component | |
| VirB10 | SalY | ABC-type antimicrobial peptide transport system, | 1 |
| | | permease component | |
| VirB9 | SalY | ABC-type antimicrobial peptide transport system, | 1 |
| | | permease component | |
| VirB10 | SecG | Preprotein translocase subunit SecG | 1 |
| VirB9 | SfcA | Malic enzyme | 2 |
| VirB10 | SmtA | SAM-dependent methyltransferases | 1 |
| VirB11 | SmtA | SAM-dependent methyltransferases | 1 |
| VirB8 | SmtA | SAM-dependent methyltransferases | 2 |
| VirD4 | SurA | Parvulin-like peptidyl-prolyl isomerase | 3 |
| VirB10 | ThdF | Predicted GTPase | 1 |
| VirB9 | ThdF | Predicted GTPase | 1 |
| VirB8 | ThdF | Predicted GTPase | 3 |
| VirB8 | TlyC | Hemolysins and related proteins containing CBS | 2 |
| | | domains | |
| VirB9 | TlyC | Hemolysins and related proteins containing CBS | 3 |
| | | domains | |
| VirB11 | TrpS | Tryptophanyl-tRNA synthetase | 1 |
| VirB8 | TrpS | Tryptophanyl-tRNA synthetase | 1 |
| VirB10 | Ttg2A | ABC-type transport system involved in resistance | 2 |
| | | to organic solvents, ATPase component | |
| VirB10 | TypA | Predicted membrane GTPase involved in stress | 1 |
| | | response | |
| VirB9 | TypA | Predicted membrane GTPase involved in stress | 1 |
| | | response | |
| VirB11 | UspA | Universal stress protein UspA and related | 1 |
| | | nucleotide-binding proteins | |
| VirB9 | UspA | Universal stress protein UspA and related | 3 |
| | | nucleotide-binding proteins | |
| VirB8 | Uup | ATPase components of ABC transporters with | 2 |
| | | duplicated ATPase domains | |
| VirB10 | Uup | ATPase components of ABC transporters with | 3 |
| | | duplicated ATPase domains | |
| VirB11 | Uup | ATPase components of ABC transporters with | 3 |
| | | duplicated ATPase domains | |
| VirB9 | Uup | ATPase components of ABC transporters with | 3 |
| | | duplicated ATPase domains | |
| VirD4 | Uup | ATPase components of ABC transporters with | 3 |
| | | duplicated ATPase domains | |
| VirB9 | UvrA | Excinuclease ATPase subunit | 3 |
| VirB10 | UvrB | Helicase subunit of the DNA excision repair | 1 |
| | | complex | |
| VirD4 | UvrC | Nuclease subunit of the excinuclease complex | 3 |
| VirB10 | VirB10 | Type IV secretory pathway, VirB10 components | 3 |
| VirB10 | VirB4 | Type IV secretory pathway, VirB4 components | 1 |
| VirB8 | VirB4 | Type IV secretory pathway, VirB4 components | 1 |
| VirD4 | VirB4 | Type IV secretory pathway, VirB4 components | 1 |
| VirB10 | VirB7 | VirB7 | 1 |
| VirB8 | VirB7 | VirB7 | 1 |
| VirB10 | VirB9 | VirB9 protein precursor | 1 |
| VirB11 | VirB9 | Type IV secretory pathway, VirB9 components | 1 |
| VirB9 | VirB9 | Type IV secretory pathway, VirB9 components | 1 |
| VirB10 | VirD4 | Type IV secretory pathway, VirD4 components | 1 |
| VirB10 | WcaA | Glycosyltransferases involved in cell wall | 3 |
| | | biogenesis | |
| VirB8 | WcaA | Glycosyltransferases involved in cell wall | 3 |
| | | biogenesis | |
| VirB8 | XerC | Integrase | 3 |
| VirB10 | YajC | Preprotein translocase subunit YajC | 2 |
| VirB9 | YhbG | ABC-type (unclassified) transport system, | 1 |
| | | ATPase component | |
| VirB8 | YhbG | ABC-type (unclassified) transport system, | 2 |
| | | ATPase component | |

TABLE 4

| gene or COG | R. sibirica ORF |
|---|---|
| ManB | rsib_orf.39 |
| AccA | rsib_orf.1142 |
| AccC | rsib_orf.1143 |
| AcoB | rsib_orf.359 |
| AcrA | rsib_orf.128 |
| AcrA | rsib_orf.241 |
| AcrB | rsib_orf.130 |
| AcrB | rsib_orf.131 |
| AcrB | rsib_orf.133 |
| AcrB | rsib_orf.495 |
| AlaS | rsib_orf.769 |
| AmpD | rsib_orf.735 |
| AraD | rsib_orf.22 |
| AspS | rsib_orf.523 |
| AtoS | rsib_orf.1151 |
| AtpA | rsib_orf.867 |
| AtpC | rsib_orf.870 |
| AtpF | rsib_orf.690 |
| BaeS | rsib_orf.107 |
| BioF | rsib_orf.792 |
| BirA | rsib_orf.10 |
| CaiC | rsib_orf.1140 |
| CcmA | rsib_orf.876 |
| CcmF | rsib_orf.1018 |
| ClpA | rsib_orf.657 |
| ClpX | rsib_orf.1030 |
| COG0319 | rsib_orf.965 |
| COG0477 | rsib_orf.165 |
| COG0477 | rsib_orf.890 |
| COG0477 | rsib_orf.327 |
| COG0477 | rsib_orf.999 |
| COG0477 | rsib_orf.1265 |
| COG0477 | rsib_orf.282 |
| COG0694 | rsib_orf.1081 |
| COG0729 | rsib_orf.505 |
| COG0742 | rsib_orf.1289 |
| COG0795 | rsib_orf.908 |
| COG1160 | rsib_orf.1069 |
| COG1189 | rsib_orf.1275 |
| COG1214 | rsib_orf.1283 |
| COG1322 | rsib_orf.1042 |
| COG1357 | rsib_orf.1290 |
| COG1373 | rsib_orf.913 |
| COG1678 | rsib_orf.671 |
| COG1999 | rsib_orf.672 |
| COG2373 | rsib_orf.1263 |
| COG2902 | rsib_orf.932 |
| COG2945 | rsib_orf.1343 |
| COG2984 | rsib_orf.204 |
| COG3177 | rsib_orf.171 |
| COG3202 | rsib_orf.31 |
| COG3202 | rsib_orf.970 |
| COG3577 | rsib_orf.758 |
| ComEC | rsib_orf.701 |
| CyoB | rsib_orf.147 |
| CysQ | rsib_orf.302 |
| DapD | rsib_orf.459 |
| Def | rsib_orf.429 |
| Def | rsib_orf.633 |
| DnaG | rsib_orf.766 |
| DnaK | rsib_orf.442 |
| DnaK | rsib_orf.470 |
| EmrA | rsib_orf.376 |
| Era | rsib_orf.644 |
| FabD | rsib_orf.983 |
| FadB | rsib_orf.1258 |
| FhlA | rsib_orf.1334 |
| FolA | rsib_orf.680 |
| FolD | rsib_orf.59 |
| FtsA | rsib_orf.367 |
| FtsI | rsib_orf.1247 |
| FumC | rsib_orf.1086 |
| GatB | rsib_orf.517 |
| GlmU | rsib_orf.1311 |
| GlnA | rsib_orf.27 |
| GlnS | rsib_orf.258 |
| GltD | rsib_orf.714 |
| GlyA | rsib_orf.960 |
| Gmk | rsib_orf.912 |
| GppA | rsib_orf.310 |
| GpsA | rsib_orf.83 |
| GyrA | rsib_orf.435 |
| GyrA | rsib_orf.614 |
| GyrB | rsib_orf.1216 |
| HemD | rsib_orf.1346 |
| HemF | rsib_orf.724 |
| HemK | rsib_orf.782 |
| HipB | rsib_orf.29 |
| HolB | rsib_orf.493 |
| HolC | rsib_orf.747 |
| HtpG | rsib_orf.793 |
| HtrB | rsib_orf.1006 |
| IbpA | rsib_orf.345 |
| IleS | rsib_orf.1147 |
| IlvA | rsib_orf.69 |
| InfA | rsib_orf.829 |
| IscA | rsib_orf.1356 |
| IscA | rsib_orf.618 |
| KdtA | rsib_orf.596 |
| LdcA | rsib_orf.151 |
| LeuS | rsib_orf.113 |
| Lgt | rsib_orf.643 |
| LipB | rsib_orf.736 |
| Lnt | rsib_orf.206 |
| LolA | rsib_orf.761 |
| Lon | rsib_orf.68 |
| LpxA | rsib_orf.713 |
| LpxB | rsib_orf.269 |
| LpxK | rsib_orf.1005 |
| LraI | rsib_orf.704 |
| LysC | rsib_orf.943 |
| MdlB | rsib_orf.280 |
| MdlB | rsib_orf.420 |
| MdlB | rsib_orf.437 |
| MesJ | rsib_orf.527 |
| MesJ | rsib_orf.649 |
| Mfd | rsib_orf.1186 |
| MiaA | rsib_orf.41 |
| MiaB | rsib_orf.857 |
| MMT1 | rsib_orf.807 |
| MrcB | rsib_orf.456 |
| Mrp | rsib_orf.547 |
| MscS | rsib_orf.642 |
| MurC | rsib_orf.371 |
| MurE | rsib_orf.1187 |
| MurF | rsib_orf.1188 |
| MutL | rsib_orf.732 |
| MutS | rsib_orf.301 |
| NlpD | rsib_orf.1004 |
| NPY1 | rsib_orf.175 |
| NrfG | rsib_orf.474 |
| NrfG | rsib_orf.593 |
| NuoD | rsib_orf.219 |
| NuoE | rsib_orf.221 |
| NuoK | rsib_orf.879 |
| NuoM | rsib_orf.324 |
| Obg | rsib_orf.789 |
| OLE1 | rsib_orf.654 |
| PaaY | rsib_orf.61 |
| PepP | rsib_orf.1353 |
| Pnp | rsib_orf.34 |
| PolA | rsib_orf.901 |
| PutP | rsib_orf.1333 |
| PyrG | rsib_orf.177 |
| QRI7 | rsib_orf.655 |
| RbfA | rsib_orf.93 |
| RecB | rsib_orf.45 |
| RecB | rsib_orf.986 |
| RecG | rsib_orf.1199 |
| RfaG | rsib_orf.124 |
| RfaG | rsib_orf.238 |
| RimM | rsib_orf.229 |
| RlpA | rsib_orf.163 |
| RlpA | rsib_orf.588 |
| RluA | rsib_orf.786 |
| Rnc | rsib_orf.553 |
| Rnd | rsib_orf.1326 |
| RnhB | rsib_orf.440 |
| Rph | rsib_orf.1128 |
| Rpll | rsib_orf.650 |
| RplS | rsib_orf.558 |
| RpoB | rsib_orf.529 |
| RpsA | rsib_orf.1372 |
| RpsD | rsib_orf.235 |
| RpsT | rsib_orf.1117 |
| Rtn | rsib_orf.372 |
| RuvA | rsib_orf.169 |
| SalX | rsib_orf.1021 |
| SalY | rsib_orf.1022 |
| SEC59 | rsib_orf.765 |
| SecG | rsib_orf.603 |
| SfcA | rsib_orf.194 |
| SmtA | rsib_orf.683 |
| Soj | rsib_orf.627 |
| SplB | rsib_orf.233 |
| SpoT | rsib_orf.328 |
| SUA5 | rsib_orf.781 |
| SucD | rsib_orf.101 |
| SurA | rsib_orf.1220 |
| TatA | rsib_orf.947 |
| ThdF | rsib_orf.931 |
| Tig | rsib_orf.790 |
| TlyC | rsib_orf.1019 |
| TlyC | rsib_orf.966 |
| TrpS | rsib_orf.1339 |
| TruA | rsib_orf.768 |
| Tsf | rsib_orf.599 |
| Ttg2A | rsib_orf.585 |
| TypA | rsib_orf.358 |
| UspA | rsib_orf.446 |
| Uup | rsib_orf.625 |
| Uup | rsib_orf.918 |
| UvrA | rsib_orf.799 |
| UvrB | rsib_orf.439 |
| UvrC | rsib_orf.1239 |
| UvrD | rsib_orf.72 |
| VirB10 | rsib_orf.313 |
| VirB11 | rsib_orf.312 |
| VirB4 | rsib_orf.887 |
| VirB7 | rsib_orf.316 |
| VirB8 | rsib_orf.315 |
| VirB8 | rsib_orf.317 |
| VirB9 | rsib_orf.314 |
| VirB9 | rsib_orf.318 |
| VirD4 | rsib_orf.311 |
| WcaA | rsib_orf.242 |
| WcaA | rsib_orf.414 |
| WecB | rsib_orf.245 |
| XerC | rsib_orf.210 |
| XerC | rsib_orf.826 |
| YajC | rsib_orf.1207 |
| YhbG | rsib_orf.38 |
| ZnuC | rsib_orf.804 |

While this invention has been particularly shown and described with references to preferred embodiments thereof, it will be understood by those skilled in the art that various changes in form and details may be made therein without departing from the scope of the invention encompassed by the appended claims.

1. An isolated nucleic acid molecule comprising SEQ ID NO: 1.

2. An isolated nucleic acid molecule which is the complement of SEQ ID NO: 1.

3. An isolated nucleic acid molecule that encodes an amino acid sequence comprising SEQ ID NO: 2.

4. An isolated nucleic acid molecule comprising a sequence that hybridizes under highly stringent conditions to SEQ ID NO: 1 or a complement of SEQ ID NO: 1.

5. An isolated nucleic acid molecule comprising a sequence that hybridizes under highly stringent conditions to a complement of SEQ ID NO: 1 and encodes a rsib_orf.1266 polypeptide.

6. A probe comprising a nucleotide sequence that comprises at least about 40 nucleotides of SEQ ID NO: 1.

7. An isolated nucleic acid comprising at least about 40 nucleotides, wherein the sequence is hybridizable to SEQ ID NO: 1.

8. An isolated polypeptide encoded by a nucleic acid comprising SEQ ID NO: 1.

9. An isolated polypeptide having an amino acid sequence comprising SEQ ID NO: 2.

10. An expression construct comprising SEQ ID NO: 1.

11. The expression construct of claim 8 wherein SEQ ID NO: 1 is operably linked to a regulatory sequence.

12. A host cell comprising the isolated nucleic acid of claim 3.

13. The host cell of claim 12 wherein the isolated nucleic acid is operably linked to a regulatory sequence.

14. A method of producing a Rickettsia sibirica rsib_orf.1266 polypeptide comprising culturing the host cell of claim 12 under conditions in which the Rickettsia sibirica rsib_orf.1266 polypeptide is produced.

15. The method of claim 14 further comprising isolating the Rickettsia sibirica rsib_orf.1266 polypeptide from the cell.

16. An isolated Rickettsia sibirica rsib-orf1266 polypeptide produced by the method of claim 15.

17. An antibody or antigen binding fragment thereof that specifically binds to a Rickettsia sibirica rsib_orf.1266 polypeptide, wherein the Rickettsia sibirica rsib_orf.1266 polypeptide is encoded by an isolated nucleic acid that encodes SEQ ID NO: 2.

18. The antibody of claim 17 wherein the antibody is a polyclonal antibody.

19. A method of identifying a nucleic acid that encodes a Rickettsia polypeptide in a sample comprising:
a) contacting the sample with a complement of a nucleotide sequence comprising SEQ ID NO: 1 under conditions in which hybridization occurs between the complement and nucleic acid in the sample using high stringency conditions;
b) identifying the nucleic acid of a) which hybridizes to the complement of the nucleotide sequence comprising SEQ ID NO: 1 under high stringency conditions,
thereby identifying a nucleic acid that encodes a Rickettsia polypeptide in a sample.

20. A method of identifying a nucleic acid that encodes a Rickettsia sibirica rsib_orf.1266 polypeptide in a sample comprising:
a) contacting the sample with a complement of a nucleotide sequence comprising SEQ ID NO: 1 under conditions in which hybridization occurs between the complement and nucleic acid in the sample using high stringency conditions;
b) identifying the nucleic acid of a) which hybridizes to the complement of the nucleotide sequence comprising SEQ ID NO: 1 under high stringency conditions,
thereby identifying a nucleic acid that encodes a Rickettsia sibirica rsib_orf.1266 polypeptide in a sample.

21. A method of identifying a Rickettsia polypeptide in a sample comprising:
a) contacting the sample with an antibody or antigen binding fragment thereof that specifically binds to a Rickettsia sibirica rsib_orf.1266 polypeptide wherein the Rickettsia sibirica rsib_orf.1266 polypeptide is encoded by an isolated nucleic acid that encodes SEQ ID NO: 2; and
b) identifying the polypeptide which specifically binds to the antibody, thereby identifying a Rickettsia polypeptide in a sample.

22. The method of claim 21 wherein the antibody is a polyclonal antibody.

23. A method of identifying a Rickettsia sibirica rsib_orf.1266 polypeptide in a sample comprising:
a) contacting the sample with an antibody or antigen binding fragment thereof that specifically binds to a Rickettsia sibirica rsib_orf.1266 polypeptide wherein the Rickettsia sibirica rsib_orf.1266 polypeptide is encoded by an isolated nucleic acid that encodes SEQ ID NO: 2; and
b) identifying the polypeptide which specifically binds to the antibody, thereby identifying a Rickettsia sibirica rsib_orf.1266 polypeptide in a sample.

24. The method of claim 23 wherein the antibody is a polyclonal antibody.

25. A method of identifying an agent that alters interaction of a polypeptide of a pathogen with a Type IV secretion system (T4SS) polypeptide, wherein the pathogen utilizes the T4SS and the polypeptide has a leucine rich repeat domain, comprising:
a) contacting a Rickettsia sibirica rsib_orf.1266 polypeptide having an amino acid sequence comprising SEQ ID NO: 2 and the Type IV secretion system polypeptide under conditions in which the Rickettsia sibirica rsib_orf.1266 polypeptide interacts with the Type IV secretion system polypeptide, with an agent to be assessed;
b) assessing the extent to which Rickettsia sibirica rsib_orf.1266 polypeptide interacts with the Type IV secretion system polypeptide in the presence of the agent to be assessed,
wherein if the extent to which Rickettsia sibirica rsib_orf.1266 polypeptide interacts with the Type TV secretion system polypeptide is altered in the presence of the agent compared to the extent to which Rickettsia sibirica rsib_orf.1266 polypeptide interacts with the Type IV secretion system polypeptide in the absence of the agent, then the agent alters interaction of a polypeptide of the pathogen with the Type IV secretion system polypeptide.

26. A method of identifying an agent that alters interaction of a Rickettsia polypeptide with a Type IV secretion system polypeptide comprising:
a) contacting a Rickettsia sibirica rsib_orf.1266 polypeptide having an amino acid sequence comprising SEQ ID NO: 2 and the Type IV secretion system polypeptide under conditions in which the Rickettsia sibirica rsib_orf.1266 polypeptide interacts with the Type IV secretion system polypeptide, with an agent to be assessed;
b) assessing the extent to which Rickettsia sibirica rsib_orf.1266 polypeptide interacts with the Type IV secretion system polypeptide in the presence of the agent to be assessed,
wherein if the extent to which Rickettsia sibirica rsib_orf.1266 polypeptide interacts with the Type IV secretion system polypeptide is altered in the presence of the agent compared to the extent to which Rickettsia sibirica rsib_orf.1266 polypeptide interacts with the Type IV secretion system polypeptide in the absence of the agent, then the agent alters interaction of a Rickettsia polypeptide with a Type IV secretion system polypeptide.

27. A method of identifying an agent that alters interaction of a Rickettsia sibirica rsib_orf.1266 polypeptide with a Type IV secretion system polypeptide comprising:
a) contacting the Rickettsia sibirica rsib_orf.1266 polypeptide having an amino acid sequence comprising SEQ ID NO: 2 and the Type IV secretion system polypeptide under conditions in which the Rickettsia sibirica rsib_orf.1266 polypeptide interacts with the Type IV secretion system polypeptide, with an agent to be assessed;
b) assessing the extent to which Rickettsia sibirica rsib_orf.1266 polypeptide interacts with the Type IV secretion system polypeptide in the presence of the agent to be assessed,
wherein if the extent to which Rickettsia sibirica rsib_orf.1266 polypeptide interacts with the Type IV secretion system polypeptide is altered in the presence of the agent compared to the extent to which Rickettsia sibirica rsib_orf.1266 polypeptide interacts with the Type IV secretion system polypeptide in the absence of the agent, then the agent alters interaction of a Rickettsia sibirica rsib_orf.1266 polypeptide with the Type IV secretion system polypeptide.

28. The method of claim 26 wherein the Type IV secretion system polypeptide is selected from the group consisting of: VirD4, VirB11 and VirB8.

29. A method of identifying an agent that alters interaction of a polypeptide of a pathogen with a Type IV secretion system (T4SS) polypeptide, wherein the pathogen utilizes the T4SS and the polypeptide has a leucine rich repeat domain comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed;
b) assessing whether apoptosis of the cell occurs,
wherein if apoptosis of the cell is altered compared to the apoptosis of a control cell, then the agent alters interaction of a Rickettsia polypeptide with Type IV secretion system polypeptide.

30. A method of identifying an agent that alters interaction of a Rickettsia polypeptide with a Type IV secretion system polypeptide comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed;
b) assessing whether apoptosis of the cell occurs,
wherein if apoptosis of the cell is altered compared to the apoptosis of a control cell, then the agent alters interaction of a Rickettsia polypeptide with Type IV secretion system polypeptide.

31. A method of identifying an agent that alters interaction of a Rickettsia sibirica rsib_orf.1266 polypeptide with a Type IV secretion system polypeptide comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed;
b) assessing whether apoptosis of the cell occurs,
wherein if apoptosis of the cell is altered compared to the apoptosis of a control cell, then the agent alters interaction of a Rickettsia sibirica rsib_orf.1266 polypeptide with Type IV secretion system polypeptide.

32. The method of claim 31 wherein the Type IV secretion system polypeptide is selected from the group consisting of: VirD4, VirB11 and VirB8.

33. A method of identifying an agent that inhibits an interaction of a polypeptide of a pathogen with a Type IV secretion system (T4SS) polypeptide, wherein the pathogen utilizes the T4SS and the polypeptide has a leucine rich repeat domain comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed;
b) assessing apoptosis of the cell,
wherein an increase in apoptosis of the cell compared to apoptosis of a control cell indicates that the agent inhibits interaction of a polypeptide of a pathogen with a Type TV secretion system (T4SS) polypeptide.

34. A method of identifying an agent that inhibits an interaction of a Rickettsia polypeptide with a Type IV secretion system polypeptide comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed;
b) assessing apoptosis of the cell,
wherein an increase in apoptosis of the cell compared to apoptosis of a control cell indicates that the agent inhibits interaction of Rickettsia polypeptide with the Type IV secretion system polypeptide.

35. A method of identifying an agent that inhibits an interaction of a Rickettsia sibirica rsib_orf.1266 polypeptide with a Type IV secretion system polypeptide comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed;
b) assessing apoptosis of the cell,
wherein an increase in apoptosis of the cell compared to apoptosis of a control cell indicates that the agent inhibits interaction of Rickettsia sibirica rsib_orf.1266 polypeptide with the Type IV secretion system polypeptide.

36. The method of claim 35 wherein the Type IV secretion system polypeptide is selected from the group consisting of: VirD4, VirB11 and VirB8.

37. A method of identifying an agent that enhances an interaction of a polypeptide of a pathogen with a Type IV secretion system (T4SS) polypeptide, wherein the pathogen utilizes the T4SS and the polypeptide has a leucine rich repeat domain comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with Type IV secretion system polypeptide in the cell, with an agent to be assessed;
b) assessing apoptosis of the cell,
wherein a decrease in apoptosis of the cell compared to apoptosis of a control cell indicates that the agent enhances interaction of a polypeptide of a pathogen with a Type IV secretion system (T4SS) polypeptide.

38. A method of identifying an agent that enhances interaction of a Rickettsia polypeptide with a Type IV secretion system polypeptide comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with the Type IV secretion system polypeptide in the cell, with an agent to be assessed;
b) assessing apoptosis of the cell,
wherein a decrease in apoptosis of the cell compared to apoptosis of a control cell indicates that the agent enhances interaction of a Rickettsia polypeptide with Type IV secretion system polypeptide.

39. A method of identifying an agent that enhances interaction of a Rickettsia sibirica rsib_orf.1266 polypeptide with a Type IV secretion system polypeptide comprising:
a) contacting a cell which comprises nucleic acid that encodes a Rickettsia sibirica rsib_orf.1266 polypeptide having an amino acid sequence comprising SEQ ID NO: 2 wherein the Rickettsia sibirica rsib_orf.1266 polypeptide, when expressed, interacts with the Type IV secretion system polypeptide in the cell, with an agent to be assessed;
b) assessing apoptosis of the cell,
wherein a decrease in apoptosis of the cell compared to apoptosis of a control cell indicates that the agent enhances interaction of a Rickettsia sibirica rsib_orf.1266 polypeptide with Type IV secretion system polypeptide.

40. The method of claim 39 wherein the Type IV secretion system polypeptide is selected from the group consisting of: VirD4, VirB11 and VirB8.

41. A method of treating an infection by a pathogen in an individual, wherein the pathogen utilizes a Type IV secretion system (T4SS), comprising administering to the individual an agent that inhibits interaction of a Rickettsia sibirica rsib_orf.1266 polypeptide with a Type IV secretion system polypeptide.

42. A method of treating a Rickettsia infection in an individual comprising administering to the individual an agent that inhibits interaction of a Rickettsia sibirica rsib_orf.1266 polypeptide with a Type IV secretion system polypeptide.

43. The method of claim 42 wherein the Rickettsia is selected from the group consisting of: Rickettsia sibirica, Rickettsia prowazekii, Rickettsia conorii, Rickettsia rickettsii and Rickettsia typhi.

44. The method of claim 42 wherein the Type IV secretion system polypeptide is selected from the group consisting of: VirD4, VirB11 and VirB8.

45. A method of inducing an immune response a pathogen in an individual, wherein the pathogen utilizes a Type IV secretion system (T4SS), comprising administering to the individual all or a portion of a Rickettsia sibirica rsib_orf.1266 polypeptide.

46. The method of claim 42 wherein the pathogen is a Rickettsia.

47. The method of claim 46 wherein the Rickettsia is selected from the group consisting of: Rickettsia sibirica, Rickettsia prowazekii, Rickettsia conorii, Rickettsia rickettsii and Rickettsia typhi.
